(19) United States
(12) Patent Application Publication
(10) Pub. No.: US 2025/0084375 A1
(43) Pub. Date: Mar. 13, 2025

(54) Three-Dimensional Skin Constructs

(71) Applicant: NUtech Ventures, Lincoln, NE (US)

(72) Inventors: Fanben Meng, Lincoln, NE (US); Ruiguo Yang, Lincoln, NE (US); Haiwei Zhai, Lincoln, NE (US); Xiaowei Jin, Lincoln, NE (US)

(73) Assignee: NUtech Ventures, Lincoln, NE (US)

(21) Appl. No.: 18/830,822

(22) Filed: Sep. 11, 2024

Related U.S. Application Data
(60) Provisional application No. 63/582,234, filed on Sep. 12, 2023.

(51) Int. Cl. C12N 5/071

(52) U.S. Cl. CPC C12N 5/0629; C12N 2502/1323; C12N 2503/02; C12N 2513/00

(57) ABSTRACT

The present disclosure describes methods of generating three-dimensional skin tissue constructs comprising a basal-to-suprabasal transition.

### CROSS-REFERENCE TO RELATED APPLICATION

This patent application claims the benefit of U.S. Provisional Patent Application No. 63/582,234, filed Sep. 12, 2023, which is incorporated by reference herein in its entirety.

### STATEMENT REGARDING FEDERALLY SPONSORED RESEARCH OR DEVELOPMENT

This invention was made with government support under P20 GM113126 awarded by the National Institutes of Health. The government has certain rights in the invention.

### BACKGROUND

Cell-cell adhesions play an important role in numerous physiological processes by maintaining the paracellular barrier, establishing intercellular communication and tuning mechanical strength. Disorders of cell-cell adhesions are associated with various diseases. For example, the skin blistering disease pemphigus is caused by epithelial cell junction disruption. In the most common and potentially fatal form of pemphigus, pemphigus vulgaris (PV), autoantibodies attack desmosomes in keratinocytes, primarily the desmosomal cadherin desmoglein 3 (Dsg3), leading to the disruption of cell-cell adhesion at the basal and suprabasal layers, as well as their interface. Studies have shown that the immunological perspective is insufficient in understanding the mechanism of the disease and guiding therapeutic support. There is strong evidence that the microenvironments of cell-cell adhesions significantly contribute, and potentially dictate, the pathogenesis of PV. However, most of these studies were conducted using two-dimensional (2D) keratinocyte monolayers. Though convenient, these monolayers often fail to reproduce important in vivo characteristics of the epidermis, such as the structural hierarchy, the geometrical complexity, and the cell-extracellular matrix (ECM) interactions.

Three-dimensional (3D)-cultured keratinocytes have been shown to behave differently from those cultured in a monolayer, especially in response to inflammation and mechanical stimulation that cause alterations of cell-cell and cell-ECM interactions. Current efforts to produce stratified epithelium by 3D bioprinting have introduced desired matrices and recreated tissue architectures in a layer-by-layer fashion. However, limited by resolutions of current 3D printing technologies, they are unable to generate a stratified structural equivalent which mimics the fine layers at basal and suprabasal locations. These deep epidermal tissue layers are often a few tens of micrometers in thickness, consisting of less than 10 layers of cells. Of interest, the basal and suprabasal layers are the targets of PV antibodies, where the antibody-induced cell-cell adhesion disruption originates. The direct cell-cell interactions across these layers are also important in the pathogenesis of blistering in PV. In vitro recapitulation of such compact cell-cell adhesions in a 3D arrangement is still a major challenge for scaffold-based biofabrication methods, as the degradation rate of the supporting matrices is typically involved to match the pace of new tissue formation. Although skin organoids produced by stem cell differentiation and self-organization could be possible solutions, these self-assembled mini organs often do not allow precise spatial control.

Hence, there is a need to construct in vitro 3D stratified tissue architectures that better mimic physiological and pathological microenvironments, allowing for increased precision in the prediction of cellular behaviors during disease progression and in response to potential therapies.

### BRIEF SUMMARY

The present disclosure provides methods of generating a three-dimensional skin tissue construct comprising a basal-to-suprabasal transition, the method comprising: (a) depositing onto a substrate a droplet comprising one or more undifferentiated keratinocyte cells; (b) depositing onto the substrate another droplet comprising one or more additional undifferentiated keratinocyte cells; and (c) maintaining the undifferentiated keratinocyte cells on the substrate in a condition that promotes differentiation of undifferentiated keratinocyte cells; wherein the droplet of (b) is deposited a predetermined distance away from the droplet of (a); wherein undifferentiated keratinocyte cells migrate from droplets of (a) and (b), generating a basal cell layer; wherein proliferation and differentiation of keratinocyte cells of the basal cell layer generate differentiated suprabasal keratinocyte cells and a basal-to-suprabasal transition between at least a subset of adjacent keratinocyte cells of the basal layer and differentiated suprabasal keratinocyte cells; and wherein the extent of differentiation of keratinocyte cells and the extent of the basal-to suprabasal transition present after (c) varies based on the predetermined distance.

The present disclosure provides methods of testing an effect of a pharmaceutical on skin tissue, the method comprising: (1) generating a three-dimensional skin tissue construct comprising a basal-to-suprabasal transition according to any of the methods described above; (2) contacting the three-dimensional skin tissue construct of (1) with a pharmaceutical; and (3) observing a change of the three-dimensional skin tissue construct after (2).

The present disclosure provides disease-state models of skin tissue, the method comprising: (1) generating a three-dimensional skin tissue construct comprising a basal-to-suprabasal transition according to the method of any one of aspects 1-8; and (2) contacting the three-dimensional skin tissue construct of (1) with an agent to alter the construct to mimic a disease state.

Additional aspects are as provided herein.

Without wishing to be bound by any particular theory, there may be discussion herein of beliefs or understandings of underlying principles relating to the materials and methods disclosed herein. It is recognized that regardless of the ultimate correctness of any mechanistic explanation or hypothesis, an aspect of the disclosure can nonetheless be operative and useful.

### BRIEF DESCRIPTION OF THE DRAWINGS

FIGS. 1A-1H: 3D-cultured keratinocytes with fibrin encapsulation. FIGS. 1A, 1B) Time-lapse Z-stacked confocal images showing the distribution of GFP-E-cad-HaCaT cells from the side (FIG. 1A) and the top-down (FIG. 1B) view over time. Scale bar: 500 μm. FIG. 1C) Plots of the distance of leading HaCaT cells versus time, demonstrating the cell migration. (mean±s.d., n≥4). FIG. 1D) Plots of cellular fluorescence intensity of HaCaT cells versus time, demonstrating the cell proliferation (normalized by intensity at day 0, mean±s.d., n≥4). FIG. 1E) Panoramic Z-stacked fluorescence images showing the collective migration of 3D-cultured HaCaT cells in the surrounding fibrin gel matrix, zone (i), from the cell source droplet, zone (ii), and the self-triggered differentiation randomly occurred in the biofabricated construct. Scale bar: 500 μm. FIG. 1F) Representative fluorescence images showing HaCaT cell distribution and differentiation status. Scale bar: 100 μm. FIG. 1G) Bar charts showing the expressions of K5 (left bar of pair of bars) and K10 (right bar of pair of bars) in zones (i) and (ii). (mean±s.d., n=3). FIG. 1H) Spatially guided construction of stratified epidermal models. Schematic image of the workflow of epidermal model fabrication. (i) Keratinocyte-laden fibrin droplets are bioprinted as initial cell sources and geographical cues. (ii) 3D cultured cells proliferate and collectively migrate to generate a confluent basal layer between cell source droplets. (iii) The defined interspace results in high cell density triggering basal-to-suprabasal differentiation and multilayer formation (SOM: self-organized multilayer).

FIGS. 2A-2D: SOM formation at an interspace created between bioprinted cell source droplets. FIG. 2A, 2B) Time-lapse panoramic Z-stacked confocal images showing the distribution of GFP-E-cad-HaCaT cells from the side (FIG. 2A) and the top-down (FIG. 2B) view over time. Scale bar: 1000 μm. FIG. 2C) Panoramic Z-stacked fluorescence images showing spatial distribution of biomarkers and dyes, demonstrating differentiation status of HaCaT cells. Scale bar: 1000 μm. FIG. 2D) Plots of fluorescence intensity measured at each pixel along the central axis of a pair of cell source droplets as illustrated by the dash lines in (c-i).

FIGS. 3A-3E: Basal-to-suprabasal transition in SOMs. FIG. 3A) Schematic image illustrating the transition of differentiation status from basal to suprabasal layers in the SOMs. FIG. 3B) 3D-reconstructed confocal images showing the spatial distribution of K5 expressing cells and K10 expressing cells in a scanned volume of 101.41 μm×101.41 μm×16.72 μm. The lateral views (bottom row) demonstrate the stratification of keratinocyte layers. FIG. 3C) Confocal images of K5/K10/DAPI-stained HaCaT cells located at three representative layers of a SOM in a bottom-up scan (five slices with a Z-step of 2.19 μm), demonstrating a vertical transition of cell differentiation statuses. Scale bar: 20 μm. FIG. 3D) Bar charts showing K5 (left-side bars of each set of bars) and K10 (right-side bars of each set of bars) stained area of each scanned layer in SOMs, demonstrating the differentiation statuses of HaCaT cells. (mean±s.d., n=3). FIG. 3E) Bar charts showing the mean nucleus area and the population density of keratinocytes located in each scanned layer in SOMs, presenting the transition of cellular patterns (mean±s.d., n=3).

FIGS. 4A-4E: Pathological changes of desmosomal junctions in response to anti-Dsg3 antibody. FIG. 4A) Confocal images of Dsg3-stained HaCaT cells at a bottom basal layer and an upper suprabasal layer of representative SOMs without (left column) and with (right column) 2 μg mL⁻¹ AK23 mAbs treatment. FIGS. 4B, 4C) Plots of Dsg3 fluorescence intensity of each pixel along a reference line versus relative distance to the line center for a representative desmosomal junction at a bottom basal layer (FIG. 4B) and an upper suprabasal layer (FIG. 4C) of a typical SOM with and without AK23 treatment. FIG. 4D) Bar charts showing full width of half max (FWHM) of representative regions at the basal layer and the suprabasal layer of SOMs with and without AK23 treatment (normalized by intensity of untreated control group, mean±s.d., n≥7, *p<0.05). FWHM value was associated with junction width. FIG. 4E) Bar charts showing the mean Dsg3 fluorescence intensity of representative regions at the basal layer and the suprabasal layer of SOMs with and without AK23 treatment (normalized by intensity of untreated control group, mean±s.d., n≥7, *p<0.05).

FIGS. 5A-5D: Influence of the fibrin concentration on 3D cultured keratinocytes. Time-lapsed brightfield images of keratinocytes (1×10⁶ cells/mL) encapsulated in fibrin gel matrices with concentrations of (FIG. 5A) 5 mg/mL, (FIG. 5B) 10 mg/mL, and (FIG. 5C) 20 mg/mL over a 10-day period. Day 0 was defined four days after fabrication. FIG. 5D) Plots of cellular area of keratinocytes and keratinocyte aggregates within fibrin matrices (at 5 mg/mL, 10 mg/mL, and 20 mg/mL) versus time (normalized by the mean of Day 0 of each group, mean±s.d., n=5), demonstrating expansion of keratinocytes through the 3D matrices, which is achieved by cell proliferation and migration. 10 mg/mL was selected to maintain the 3D microenvironments for keratinocytes in this study based on the observation of the most rapid cell expansion among three gel concentrations. Scale bar: 500 μm.

FIGS. 6A-6E: Further tuning fibrin gel stability with addition of aprotinin. Time-lapse brightfield images of keratinocytes encapsulated in 10 mg/ml fibrin gel matrices (FIG. 6A) without aprotinin (−−−) and with 25 μg/mL aprotinin treatments in (FIG. 6B) only the cell-laden droplet (−+−), (FIG. 6C) only the surrounding matrix (+−−), (FIG. 6D) both the droplet and the surrounding matrix (++−), and (FIG. 6E) the culture media (+++). To promote collective migration of keratinocytes in the surrounding fibrin matrix, the option of FIG. 6E was used in this study. Scale bar: 500 μm.

FIG. 7: Collective migration of keratinocytes. Keratinocytes proliferated and collectively migrated in the surrounding matrix, Zone (i) from the cell source droplet, Zone (ii), as shown in the brightfield image and fluorescence images with K5 (marker for highly proliferative basal cells) and K10 (marker for differentiated suprabasal cells) staining. The keratinocyte differentiation was mainly observed in Zone (II). Scale bar: 100 μm.

FIGS. 8A and 8B: Spatial patterns of 3D-cultured keratinocytes. Representative fluorescence images of GFP-E-cad-HaCaT showing the spatial arrangement of the epidermal cells that (FIG. 8A) migrated out of and (FIG. 8B) aggregates within the cell source droplet. The distinctive distribution of E-cad that indicated spatial patterns of keratinocytes was observed in different sample regions. Scale bar: 50 μm.

FIGS. 9A and 9B: Projection and stitch of confocal images. FIG. 9A) A series of fluorescence images showing the Z-stack of three slices from a vertical multi-channel scan with a Z-step size of 30.02 μm. Scale bar: 100 μm. FIG. 9B) A series of fluorescence images (tiles for the panoramic images of FIG. 2B Day 4) showing the stitch of fifteen tiles with 10% overlap. Scale bar: 1000 μm.

FIGS. 10A and 10B: K10 expression in the biofabricated tissue constructs with a pair of cell-laden droplets as geographical cues. Fluorescence images showing K10 expression of keratinocytes located (FIG. 10A) in the interspace between two cell source droplets and (FIG. 10B) within a cell source droplet. Compared with single-droplet constructs (FIG. 1F), the K10 expression within droplets showed a similar pattern while cells that migrated out of droplets significantly enhanced the expression of the differentiation marker in the interspace with the presence of the geographical cue. Scale bar: 100 μm.

FIGS. 11A and 11B: Stratification of SOMs. FIG. 11A) a 3D reconstructed confocal image showing the lateral view of a typical SOM. Scan area: 101.41 μm×101.41 μm×16.72 μm. FIG. 11B) Box bar charts showing the measured layer thickness of the total vertical scan, basal and suprabasal layers, respectively (box: mean±s.d., error bar: min-max, n: 14 representative regions of three independent samples).

FIGS. 12A and 12B: Illustration of FWHM quantification. FIG. 12A) Fluorescence image of Dsg3-stained keratinocytes indicating the desmosomal disassembly after a 24-hour treatment of AK23 mAb (2 μg/mL). The white reference line bridges the nuclei of two adjacent cells at a junction site. Scale bar: 20 μm. FIG. 12B) Plots of Dsg3 fluorescence intensity vs pixel number along the reference line. Iₘₐₓ: Absolute peak intensity; I_{b}: Background noise that is defined as the average detected intensity of first 5 pixels at each end of the reference line (I_{b1} and I_{b2}); Iₘₐₓ−I_{b}: Relative peak intensity; I_{half max}: Half of the relative peak intensity; Xₘₐₓ: Maximum pixel number where I_{half max} is reached; Xₘᵢₙ: Minimum pixel number where I_{half max} is reached; FWHM: Xₘₐₓ−Xₘᵢₙ.

FIG. 13: Spatially guided construction of 3D skin models with stratified epidermal layers and a vascularized dermal layer. A schematic image of the workflow for fabricating the model, showing keratinocyte-laden fibrin droplets deposited on a human dermal fibroblast (HDF)-laden fibrin hydrogel, where the hydrogel is perfusable with an engineered microvascular channel.

FIG. 14: Time-lapse brightfield images showing the proliferation and migration of HaCaT cells (top row) and the proliferation of HDF (bottom row). Scale bar: 1000 μm.

FIG. 15: Intermediate stage of vascularized skin models, revealed by a 3D-reconstructed confocal image (top) and panoramic Z-stacked fluorescence images (bottom). Fluorescence channels showing migrating basal keratinocytes of GFP-E-cad-HaCaT before forming a confluent basal layer (E-cad); HDFs embedded in the dermal layer (α-SMA); endothelialized microvasculature (CD31); and overall cell distribution within the model (DAPI). Scale bar: 1000 μm.

FIG. 16: Mature stage of vascularized skin models revealed by a 3D-reconstructed confocal image and panoramic Z-stacked fluorescence images, showing the formation of confluent epidermal cell layers on the vascularized dermal layer. Scale bar: 1000 μm.

FIGS. 17A-17C: Immunohistochemistry staining of the biofabricated skin tissue. Sectional views of the skin models highlighting: FIG. 17A) The microvasculature channel as an endothelial barrier (CD 31: endothelial marker, COL-IV: collagen type-IV, basement membrane marker); FIG. 17B) The stratified epidermis with highly differentiated suprabasal keratinocytes (co-localization of K10 and involucrin, INV in keratinocytes suggesting the transition from the stratum spinosum to the stratum granulosum in the suprabasal layers); and FIG. 17C) Dermal ECM remodeling (co-localization of a-SMA and COL-I indicating the deposition of collagen type-I within the fibrin hydrogel by the embedded HDFs).

FIGS. 18A and 18B: Dynamic ECM remodeling in the dermal layer by fibroblasts. FIG. 18A) Time-lapse fluorescent images demonstrating the proliferation and activation of resident HDFs (with high expression of α-SMA) and the deposition of collagen type-I (COL-I), a major component of dermal ECM. Scale bar: 400 μm; and FIG. 18B) Box charts quantitatively showing ECM remodeling by HDFs, revealed by the increasing matrix viscoelasticity (box: 25th-75th percentile; whiskers: mean±s.d., n=18).

FIGS. 19A and 19B: The influence of tissue barriers on molecular diffusion in the constructed dermal layer. FIG. 19A) Time-lapse fluorescent images showing the diffusion of dextran (150 kDa) in fibrin-based dermal layers with different matrix components, including pure fibrin, a fibroblast-induced stromal barrier, an endothelial barrier along the microchannel, and both tissue barriers. Scale bar: 500 μm; and FIG. 19B) Bar graphs quantitatively showing the reduced diffusional permeability of the dermal layer when stromal and/or endothelial barriers are integrated, demonstrating the impact of tissue barriers on molecular diffusion (mean±s.d., n ≥3, *p<0.05, **p<0.01).

FIGS. 20A-20C: 3D PV models used to study the impact of autoimmune antibody transport on PV pathogenesis. FIG. 20A) Confocal microscope images of microsectioned tissue slices from the PV skin models showing the distribution of autoimmune antibody (AK23) and Dsg3 cell-cell junctions within the stratified epidermis. Two AK23 treatment routes were compared: 1) direct application onto the epidermis, and 2) injection via the microvascular channel to mimic in vivo molecular delivery. Scale bar: 20 μm; FIG. 20B) Bar graphs quantitatively showing AK23 density and the co-localization of AK23 and Dsg3, illustrating the impact of dermal microenvironments on antibody penetration (mean±s.d., n≥3, *p<0.05, **p <0.01, ***p<0.001, n.s.: not significant); and FIG. 20C) Bar charts displaying the full-width at half-maximum (FWHM) of Dsg3 at basal-to-basal and basal-to-suprabasal junctions, demonstrating junction dissociation in response to AK23 through the different treatment routes (mean±s.d., n≥4, *p<0.05, **p<0.01, ***p<0.001, n.s.: not significant).

### DETAILED DESCRIPTION

The present disclosure provides methods of generating a three-dimensional skin tissue construct comprising a basal-to-suprabasal transition, the method comprising: (a) depositing onto a substrate a droplet comprising one or more undifferentiated keratinocyte cells; (b) depositing onto the substrate another droplet comprising one or more additional undifferentiated keratinocyte cells; and (c) maintaining the undifferentiated keratinocyte cells on the substrate in a condition that promotes differentiation of undifferentiated keratinocyte cells; wherein the droplet of (b) is deposited a predetermined distance away from the droplet of (a); wherein undifferentiated keratinocyte cells migrate from droplets of (a) and (b), generating a basal cell layer; wherein proliferation and differentiation of keratinocyte cells of the basal cell layer generate differentiated suprabasal keratinocyte cells and a basal-to-suprabasal transition between at least a subset of adjacent keratinocyte cells of the basal layer and differentiated suprabasal keratinocyte cells; and wherein the extent of differentiation of keratinocyte cells and the extent of the basal-to suprabasal transition present after (c) varies based on the predetermined distance.

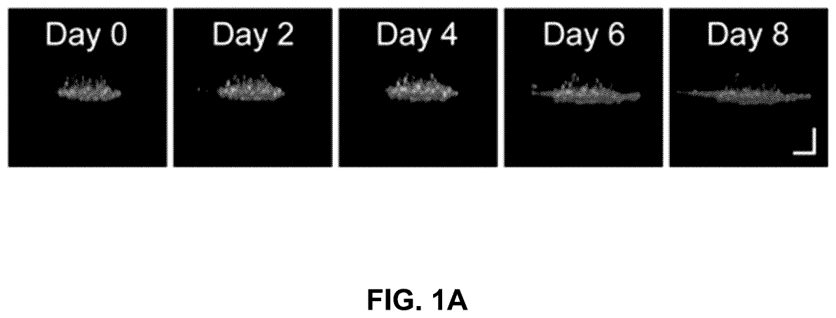

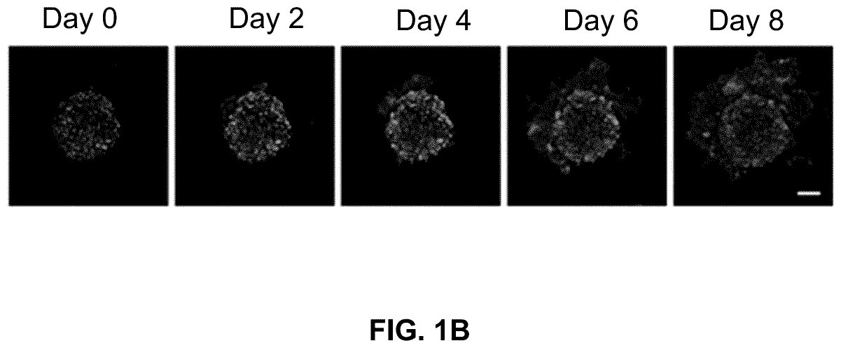

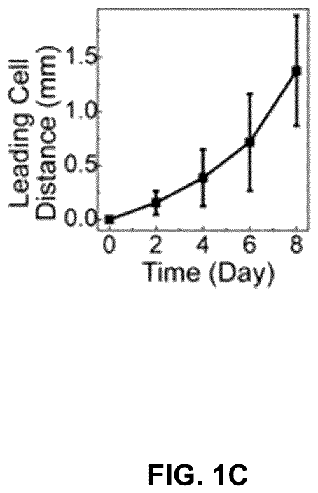

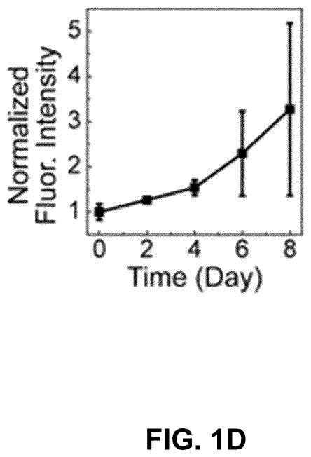

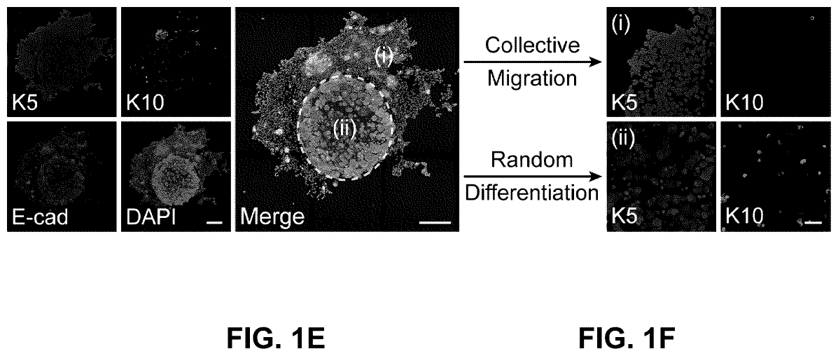

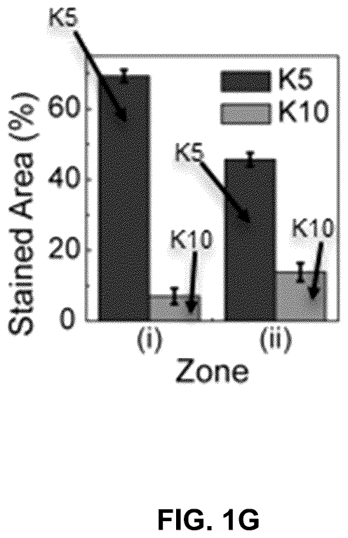

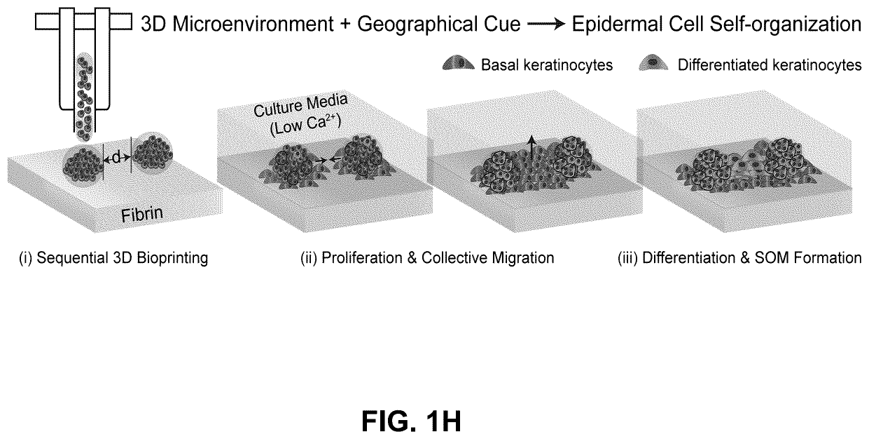

As used herein, an undifferentiated cell is a cell in any state prior to full differentiation of the cell, i.e., where the cell can further differentiate. Basal keratinocytes are undifferentiated keratinocyte cells. Unlike conventional stem cell differentiation, the basal keratinocytes gradually change their characteristics and functions during basal-to-suprabasal differentiation or stratification. From bottom to top, keratinocytes gradually lose their ability to proliferate but enhance their rigidity as protective barriers. Therefore, several differentiated states of keratinocytes can be observed in suprabasal layers under physiological conditions and in the models described herein. Fully differentiated keratinocytes, usually referred to as corneocytes, are located in the outermost layer of the epidermis. These fully differentiated keratinocytes have lost their nuclei and cannot proliferate. Under the conditions described in the Examples below, the generation of corneocytes was not observed. The differentiated keratinocytes in FIG. 1H represent suprabasal keratinocytes that differentiate from basal keratinocytes.

Without wishing to be bound by theory, it is believed that keratinocyte differentiation is triggered by high cell density, e.g., in the formation of a highly compact basal cell layer. However, generating a highly compact basal cell layer in vitro is very challenging. The methods described herein provide a solution to address this challenge and promote keratinocyte differentiation without introducing any external chemical factors. Without wishing to be bound by theory, it is believed that wherever a high-cell-density region forms, keratinocyte differentiation and the basal-to-suprabasal transition will be automatically triggered, e.g., when cells collectively migrate from the droplets to form a confluent monolayer in the predefined space between the droplets, where due to spatial constraints, the basal cells within the interspace cannot expand horizontally and therefore begin to expand vertically. The basal-to-suprabasal transition is driven by proliferation of undifferentiated basal cells and differentiation of basal keratinocyte cells.

The droplet-to-droplet distance determines how soon a compact basal layer will form or whether an intact basal layer will form. If the distance varies, the coverage of basal keratinocytes on the substrate will be affected, thereby influencing the extent of keratinocyte differentiation and the basal-to-suprabasal transition. Without wishing to be bound by theory, it is believed that if the distance decreases, a smaller stratified epidermal area will be obtained. Without wishing to be bound by theory, it is believed that if the distance increases, more time will be required for cells to migrate and proliferate in order to form a compact basal cell layer. The increased travel distance may raise the likelihood of generating high-density regions before an intact basal layer forms between the droplets. These high-density regions could undergo self-triggered differentiation, reducing keratinocyte proliferation.

In aspects, the predetermined distance is 800 to 1000 micrometers measured edge-to-edge from the droplets of (a) and (b) deposited on the substrate. In aspects, the predetermined distance is 500 to 2000 micrometers, 600 to 2000 micrometers, 700 to 2000 micrometers, 800 to 2000 micrometers, 900 to 2000 micrometers, 1000 to 2000 micrometers, 1100 to 2000 micrometers, 1200 to 2000 micrometers, 1300 to 2000 micrometers, 1400 to 2000 micrometers, 1500 to 2000 micrometers, 1600 to 2000 micrometers, 1700 to 2000 micrometers, 1800 to 2000 micrometers, 1900 to 2000 micrometers, 500 to 1900 micrometers, 600 to 1900 micrometers, 700 to 1900 micrometers, 800 to 1900 micrometers, 900 to 1900 micrometers, 1000 to 1900 micrometers, 1100 to 1900 micrometers, 1200 to 1900 micrometers, 1300 to 1900 micrometers, 1400 to 1900 micrometers, 1500 to 1900 micrometers, 1600 to 1900 micrometers, 1700 to 1900 micrometers, 1800 to 1900 micrometers, 500 to 1800 micrometers, 600 to 1800 micrometers, 700 to 1800 micrometers, 800 to 1800 micrometers, 900 to 1800 micrometers, 1000 to 1800 micrometers, 1100 to 1800 micrometers, 1200 to 1800 micrometers, 1300 to 1800 micrometers, 1400 to 1800 micrometers, 1500 to 1800 micrometers, 1600 to 1800 micrometers, 1700 to 1800 micrometers, 500 to 1700 micrometers, 600 to 1700 micrometers, 700 to 1700 micrometers, 800 to 1700 micrometers, 900 to 1700 micrometers, 1000 to 1700 micrometers, 1100 to 1700 micrometers, 1200 to 1700 micrometers, 1300 to 1700 micrometers, 1400 to 1700 micrometers, 1500 to 1700 micrometers, 1600 to 1700 micrometers, 500 to 1600 micrometers, 600 to 1600 micrometers, 700 to 1600 micrometers, 800 to 1600 micrometers, 900 to 1600 micrometers, 1000 to 1600 micrometers, 1100 to 1600 micrometers, 1200 to 1600 micrometers, 1300 to 1600 micrometers, 1400 to 1600 micrometers, 1500 to 1600 micrometers, 500 to 1500 micrometers, 600 to 1500 micrometers, 700 to 1500 micrometers, 800 to 1500 micrometers, 900 to 1500 micrometers, 1000 to 1500 micrometers, 1100 to 1500 micrometers, 1200 to 1500 micrometers, 1300 to 1500 micrometers, 1400 to 1500 micrometers, 500 to 1400 micrometers, 600 to 1400 micrometers, 700 to 1400 micrometers, 800 to 1400 micrometers, 900 to 1400 micrometers, 1000 to 1400 micrometers, 1100 to 1400 micrometers, 1200 to 1400 micrometers, 1300 to 1400 micrometers, 500 to 1300 micrometers, 600 to 1300 micrometers, 700 to 1300 micrometers, 800 to 1300 micrometers, 900 to 1300 micrometers, 1000 to 1300 micrometers, 1100 to 1300 micrometers, 1200 to 1300 micrometers, 500 to 1200 micrometers, 600 to 1200 micrometers, 700 to 1200 micrometers, 800 to 1200 micrometers, 900 to 1200 micrometers, 1000 to 1200 micrometers, 1100 to 1200 micrometers, 500 to 1100 micrometers, 600 to 1100 micrometers, 700 to 1100 micrometers, 800 to 1100 micrometers, 900 to 1100 micrometers, 1000 to 1100 micrometers, 500 to 1000 micrometers, 600 to 1000 micrometers, 700 to 1000 micrometers, 800 to 1000 micrometers, 900 to 1000 micrometers, 500 to 900 micrometers, 600 to 900 micrometers, 700 to 900 micrometers, 800 to 900 micrometers, 500 to 800 micrometers, 600 to 800 micrometers, 700 to 800 micrometers, 500 to 700 micrometers, 600 to 700 micrometers, or 500 to 600 micrometers, measured edge-to-edge from the droplets of (a) and (b) deposited on the substrate.

The 800-1000 micrometer can generate a roughly 1 mm² area of stratified epidermal layers within a 2-week testing window (first week: confluent basal layer formation; second week: basal-to-suprabasal differentiation and PV pathogenesis). Given the cell migration velocity (˜1 mm per week) under the culture conditions and cell viability (greater than three weeks), a basal cell layer should form to cover a 2000-micrometer gap between droplets in around two weeks.

Any number of droplets may be used in the methods described herein, wherein each droplet is deposited at the same predetermined distance from any other droplet.

In aspects, maintaining the undifferentiated keratinocyte cells during (c) comprises maintaining the undifferentiated keratinocyte cells in low-calcium medium. Before a highly compact basal cell layer is formed, a low-calcium culture medium (e.g., 0.35-0.4 mM calcium) cannot promote differentiation and instead maintains the undifferentiated state of keratinocyte cells. Typically, keratinocyte differentiation is triggered when the calcium concentration exceeds 1.8 mM.

In aspects, the droplets of (a) and (b) each comprise a hydrogel comprising one or more undifferentiated keratinocyte cells. In aspects, the substrate comprises a hydrogel. Commonly used ECM-based hydrogels for biofabrication may be employed to encapsulate cells, such as collagen (which can have poor printability), hyaluronic acid (which can have chemical modification for gel crosslinking), and gelatin methacryloyl (which can have chemical modification and a photoinitiator for gel crosslinking). In aspects, the hydrogel comprises fibrin. Fibrin is preferred since it is the native temporary scaffold for tissue regeneration.

In aspects, the substrate does not comprise a lattice structure. As used herein, a lattice structure of a substrate is a structure having regularly repeating units. As used herein, a lattice structure does not include vasculature. Fibrin gel is a complex, mesh-like 3D network formed by fibrin protein fibers, and as a result, no lattice structure is observed in fibrin gels.

In aspects, the substrate comprises one or more human dermal fibroblast cells. Additional dermal cell types, such as immune cells (e.g., macrophages and mast cells), adipocytes, and nerve cells (e.g., Schwann cells), can potentially be added. To better mimic the complex extracellular matrix of the dermal layer, collagen, elastin, and hyaluronic acid can be mixed with the fibrin gel.

In aspects, the substrate comprises one or more microvascular channels. Vasculature can deliver oxygen and nutrients and/or remove waste from surrounding tissues. As shown in Example 2, the integration of microvascular channels mimicked the physiological routes and barriers for molecular transport, such as the delivery of pharmaceuticals and antibodies used in the study.

The present disclosure provides methods of testing an effect of a pharmaceutical on skin tissue, the method comprising: (1) generating a three-dimensional skin tissue construct comprising a basal-to-suprabasal transition according to any of the methods described above; (2) contacting the three-dimensional skin tissue construct of (1) with a pharmaceutical; and (3) observing a change of the three-dimensional skin tissue construct after (2).

The present disclosure provides disease-state models of skin tissue, the method comprising: (1) generating a three-dimensional skin tissue construct comprising a basal-to-suprabasal transition according to the method of any one of aspects 1-8; and (2) contacting the three-dimensional skin tissue construct of (1) with an agent to alter the construct to mimic a disease state.

In aspects, the substrate comprises one or more microvascular channels and wherein the contacting of (2) comprises delivering the agent by one or more microvascular channels in the substrate.

The following are aspects of the disclosure.
1. A method of generating a three-dimensional skin tissue construct comprising a basal-to-suprabasal transition, the method comprising:
   (a) depositing onto a substrate a droplet comprising one or more undifferentiated keratinocyte cells;
   (b) depositing onto the substrate another droplet comprising one or more additional undifferentiated keratinocyte cells; and
   (c) maintaining the undifferentiated keratinocyte cells on the substrate in a condition that promotes differentiation of undifferentiated keratinocyte cells;
   wherein the droplet of (b) is deposited a predetermined distance away from the droplet of (a);
   wherein undifferentiated keratinocyte cells migrate from droplets of (a) and (b), generating a basal cell layer;
   wherein proliferation and differentiation of keratinocyte cells of the basal cell layer generate differentiated suprabasal keratinocyte cells and a basal-to-suprabasal transition between at least a subset of adjacent keratinocyte cells of the basal layer and differentiated suprabasal keratinocyte cells; and
   wherein the extent of differentiation of keratinocyte cells and the extent of the basal-to suprabasal transition present after (c) varies based on the predetermined distance.
2. The method of aspect 1, wherein the predetermined distance is 800 to 1000 micrometers measured edge-to-edge from the droplets of (a) and (b) deposited on the substrate.
3. The method of aspect 1 or 2, wherein maintaining the undifferentiated keratinocyte cells during (c) comprises maintaining the undifferentiated keratinocyte cells in low-calcium medium.
4. The method of any one of aspects 1-3, wherein the droplets of (a) and (b) each comprise a hydrogel comprising one or more undifferentiated keratinocyte cells.
5. The method of any one of aspects 1-4, wherein the substrate comprises a hydrogel.
6. The method of any one of aspects 1-5, wherein the substrate does not comprise a lattice structure.
7. The method of any one of aspects 1-6, wherein the substrate comprises one or more human dermal fibroblast cells.
8. The method of any one of aspects 1-7, wherein the substrate comprises one or more microvascular channels.
9. A method of testing an effect of a pharmaceutical on skin tissue, the method comprising:
   (1) generating a three-dimensional skin tissue construct comprising a basal-to-suprabasal transition according to the method of any one of aspects 1-8;
   (2) contacting the three-dimensional skin tissue construct of (1) with a pharmaceutical; and
   (3) observing a change of the three-dimensional skin tissue construct after (2).
10. A method of generating a disease-state model of skin tissue, the method comprising:
   (1) generating a three-dimensional skin tissue construct comprising a basal-to-suprabasal transition according to the method of any one of aspects 1-8; and
   (2) contacting the three-dimensional skin tissue construct of (1) with an agent to alter the construct to mimic a disease state.
11. The method of aspect 9 or 10, wherein the substrate comprises one or more microvascular channels and wherein the contacting of (2) comprises delivering the agent by one or more microvascular channels in the substrate.

It shall be noted that the preceding are merely examples of aspects of the disclosure. Other exemplary aspects are apparent from the entirety of the description herein. It will also be understood by one of ordinary skill in the art that each of these aspects may be used in various combinations with the other aspects provided herein.

The following example further illustrates aspects of the disclosure, but, of course, should not be construed as in any way limiting its scope.

### Example 1

### Materials and Methods

### Cell Reconstruction and Maintenance

A fully assembled GFP-E-cad was generated via inserting the GFP-E-cad cDNA into the LZBob-neo-vector, which is a modified LZRS-ms-neo-vector with multiple cloning sites for increasing cDNA fragment [Ref. 50]. The constructs were transfected into phoenix 293 cells for packaging and amplifying. Phoenix 293 cells were then cultured in medium which was prepared by Dulbecco's Modified Eagle's Medium (DMEM) (Cat. No. 11965092, ThermoFisher Scientific) supplemented with 10% fetal bovine serum, 1% penicillin and 1% GlutaMAX, for more than 2 days. Viral conditioned culture medium was collected and filtered with a 0.45 μm syringe filter. HaCaT cells were infected by culturing in the viral conditioned medium with 4 μg mL⁻¹ polybrene (Cat. No. 28728-55-4, Sigma) for 7 h at 37° C. After that, infected HaCaT cells were selected by low calcium medium with 500 μg mL⁻¹ G418 (Geneticin) until cells were healthy with stable proliferation. Low calcium medium was made by replacing DMEM with DMEM with no calcium (Cat. No. 21068028, ThermoFisher Scientific). To maintain the undifferentiated state, HaCaT cells were cultured in low calcium medium at 37° C. supplied with 5% CO₂ to ˜70% confluency. Then, cells were harvested and resuspended to a concentration of 5×10⁶ cells mL⁻¹.

### 3D Biofabrication of Epidermal Models

A typical bioink of the supporting fibrin matrix consisted of 10 mg mL⁻¹ fibrinogen (Cat. No. 341576, Millipore), 0.025 mg mL⁻¹ aprotinin (Cat. No. A4529, Millipore), and 10% v/v glycerol (Cat. No. G2025, Millipore) in the low calcium culture medium. The ink solution was freshly prepared before each fabrication. 1 unit mL⁻¹ thrombin was added to crosslink the matrix. The supporting matrix was first printed or cast to each well of a glass-bottom well plate. Two 1.5 μL hemispherical fibrin gels seeded with 1.5×10³ GFP-E-cad-HaCaT cells as cell sources were sequentially printed onto the supporting matrix with a controllable distance between 0.8 to 1 mm. The bioprinting process was conducted using a custom-built 3D bioprinter as reported in previous studies [Refs. 34-36]. The printed samples were cultured for 4 days before imaging, which allowed cells to adapt to the hydrogel matrices. 25 μg mL⁻¹ aprotinin was added to the culture medium to stabilize the fibrin matrix [Ref. 30], and the culture medium was changed with a 4-day interval.

### Anti-Dsg3 Antibody Treatment

First, the calcium concentration in culture media for both the control and the AK23-treated groups was increased to 1.8 mM to induce the formation of Ca-dependent intercellular adhesions. After culturing overnight in the high calcium culture medium, 2 μg mL⁻¹ AK23 antibody was added to the samples in the treatment group for another 24 hours.

### Immunostaining

Samples were first washed with DPBS and fixed with 4% paraformaldehyde for 45 min. Then, 0.1% Triton X-100 was used to permeabilize the samples for 1 h at room temperature. A block solution was prepared using 1% BSA and 22.52 mg mL⁻¹ glycine in DPBST (DPBS+0.1% Tween 20). Primary antibodies were introduced after the samples were blocked for 1 h. After incubating with primary antibodies at ambient temperature for 2 h, the samples were treated with secondary antibodies in 1% BSA solution overnight at 4° C. Before imaging, samples were also counterstained with DAPI for 2 h. Samples were washed at least three times after each step described above. The details of primary and secondary antibodies are listed in Table 1.

TABLE 1
Summary of immunostaining reagents

| | | Host Species & | | | Dilution |
|---|---|---|---|---|---|
| Number | Antibody or dye | Reactivity | Manufacturer | Catalog # | Ratio |
| 1 | Anti-Cytokeratin 5 | Mouse anti- | Thermo Fisher | MA5- | 1:250 |
| | antibody | human | Scientific | 12596 | |
| 2 | Anti-Cytokeratin | Rabbit anti- | Abcam | ab76318 | 1:100 |
| | 10 antibody | human | | | |
| 3 | Anti-Desmoglein | Mouse anti- | Abcam | ab231309 | 1:100 |
| | 3/PVA antibody | human | | | |
| 4 | Alexa Fluor ™ 555 | Goat anti-rabbit | Thermo Fisher | A21430 | 1:100 |
| | | | Scientific | | |
| 5 | Alexa Fluor Plus | Goat anti-mouse | Thermo Fisher | A32728 | 1:100 |
| | 647 | | Scientific | | |
| 6 | DAPI | DNA | Thermo Fisher | 62247 | 1:500 |
| | | | Scientific | | |

### Imaging Acquisition and Processing

Tissue constructs were imaged using a confocal microscope (LSM800, Zeiss) equipped with an incubation chamber for time-lapsed observation. Spectral lasers with wavelengths of 405 nm, 488 nm, 561 nm, and 633 nm were used for scanning of the four fluorescent channels. Stitch and Z-stack were performed in Zen Blue software that was associated with the microscope. ImageJ was used to generate composite microscopy images by combining fluorescent channels, maximum orthogonal (XY) projection, and 3D rendering and visualization. To minimize the effect of background noise, background subtraction was performed on all raw images before the analysis of fluorescent intensities. All related parameters, including image size, laser power, master gain, and objective pinhole diameter, were optimized for each dye and kept consistent between groups.

### Analysis of Cell Migration and Proliferation

Multitiles of Z-stacked images of 3D tissue constructs were acquired and stitched. A maximum orthogonal (XY) projection of each sample was used for the fluorescent analysis that was conducted with ImageJ. The leading cell that was the furthest distance from the initial boundary of the cell source droplet was tracked at each recording time point. Meanwhile, the summed fluorescence intensity of GFP-tagged cells was obtained to demonstrate proliferation of HaCaT cells.

### Fluorescence Intensity Analysis

Fluorescence images were analyzed with ImageJ using the “Analyze Particles” function for each fluorescent channel. For K5 and K10, total particle area and particle area percentage were obtained. For DAPI, average particle area and particle number were calculated to represent average nucleus area and cell population.

### Dsg3 Mean Intensity and Full Width at Half Maximum (FWHM) Analysis

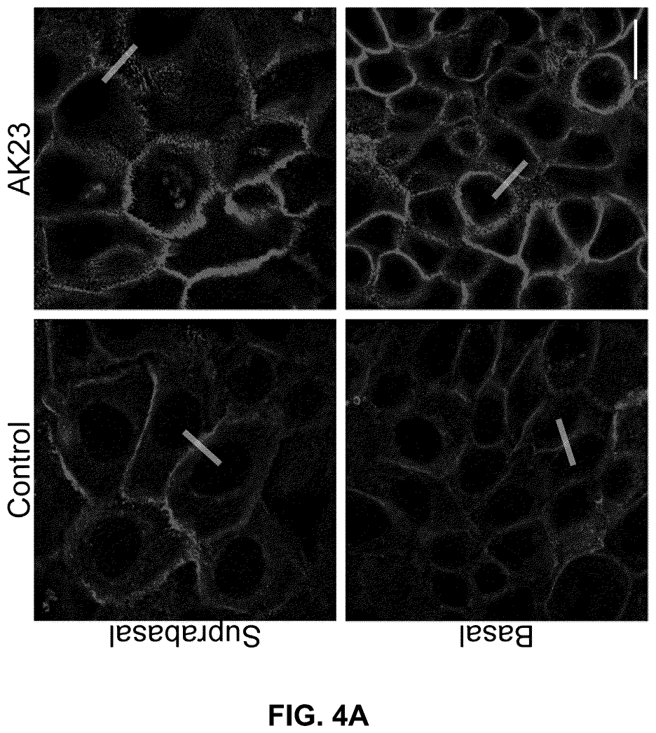

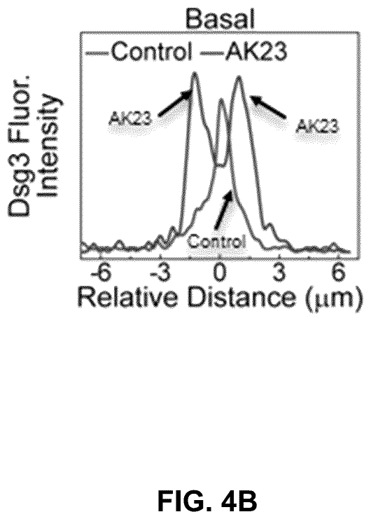

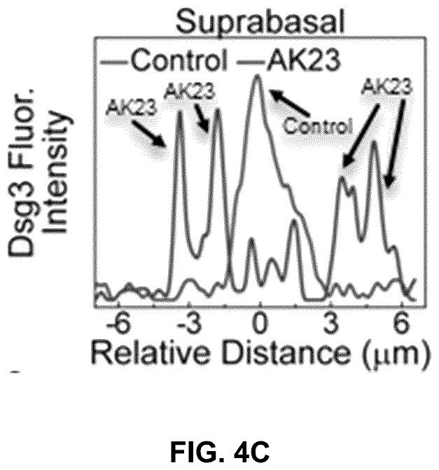

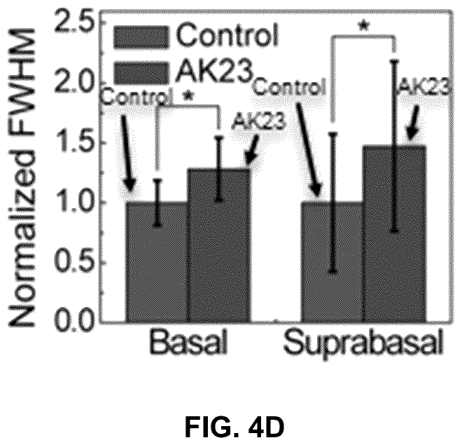

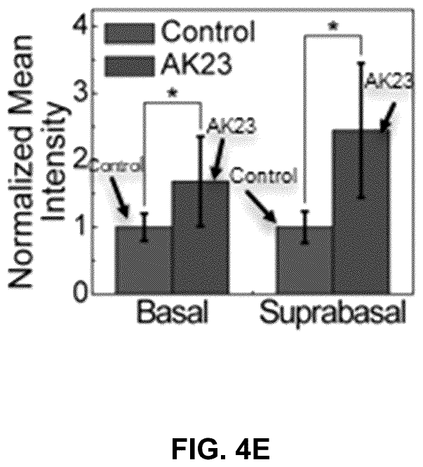

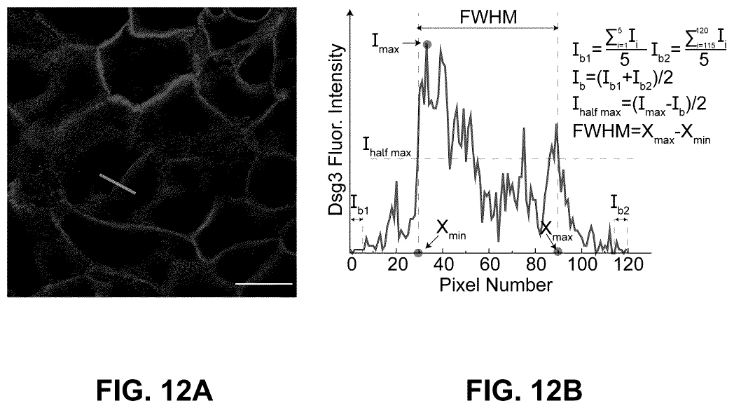

The analysis was performed by customized MATLAB scripts. Distribution curves as shown in FIGS. 12A and 12B were plotted by the Dsg3 fluorescent intensity along the line perpendicular to the cell-cell junction. Specifically, a reference line was first drawn bridging the nuclei of two adjacent cells. Dsg3 fluorescent intensities along the reference line were recorded. Based on the microscope camera setting, the pixel number could be converted to relative distance as 0.198 μm pixel⁻¹ as shown in FIGS. 4B and 4C. Then, the FWHM was calculated as depicted in FIG. 12B to quantify the mean width of a cell-cell junction. Background noise was subtracted based on the average detected intensity of first five pixels at each end of the reference line for the calculation of main peak intensity (Iₘₐₓ−I_{b}). The maximum (Xₘₐₓ) and minimum (Xₘᵢₙ) pixel numbers corresponding to half of each main peak intensity (I_{half max}) were located. FWHM was finally calculated as the difference of Xₘₐₓ and Xₘᵢₙ. The mean intensity of a single fluorescent image was calculated from the average intensity of the effective pixels. The effective pixel was defined as the pixel with a detectable intensity. Data that was shown in FIGS. 4D and 4E was normalized by the untreated control group.

### Statistical Analysis

Statistical data was analyzed using Origin (data analysis and plotting software). All data of plots and bar charts were presented as quantitative values, shown as mean±standard deviation, from n≥3 independent samples per group of experiments, as stated in the figure captions. Quantile-quantile (Q-Q) plot was used for normality test. Differences between control and treatment groups were analyzed using unpaired student t-test and Mean-Whitney U-test if normality was not met. A p-value of less than 0.05 was considered statistically significant.

### Results and Discussion

The construction of multilayered epidermal tissues combines 3D bioprinting and guided cell self-organization with the conceptual design illustrated in FIG. 1H. Optimized epidermal bioinks are sequentially deposited using a custom-built 3D printer with a spatial control over cell sources. The chemical composition, cell-loading density, and biodegradability of the bioprinted constructs are finely tuned to provide a 3D microenvironment promoting rapid proliferation and collective migration of keratinocytes. The interspace (d) between cell sources is precisely defined to create geographical cues guiding the formation of compact basal layers and the ensuant basal-to-suprabasal differentiation, which leads to the regeneration of hierarchical epidermal architectures.

### 3D Keratinocyte Culture Through Fibrin Encapsulation

To understand important cell activities including keratinocyte proliferation, migration, and differentiation in 3D microenvironments, a conventional hydrogel-scaffold method was used to build a culture platform for keratinocytes. Here, HaCaT cells, a widely used human keratinocyte line, were encapsulated in a 3D hydrogel matrix. The HaCaT cells were engineered to express green fluorescent protein (GFP)-tagged E-Cadherin (GFP-E-cad) for real-time imaging. Natural fibrin was selected as the scaffold material, owing to its well-known biocompatibility and biodegradability. Fibrin is also an important material of hemostatic plugs to assist skin regeneration during wound healing. In a typical experiment, 1×10⁶ cells mL⁻¹ of HaCaT cells were first encapsulated in a 1.5 μL fibrin hydrogel at a concentration of 10 mg mL⁻¹. Then, the cell-laden droplet was placed on bulk supporting fibrin hydrogel (˜150 μL). The proliferation and migration of encapsulated keratinocytes from the spatially defined primary site was subsequently tracked and characterized.

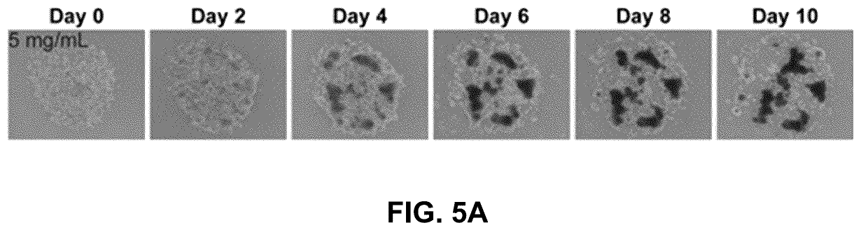

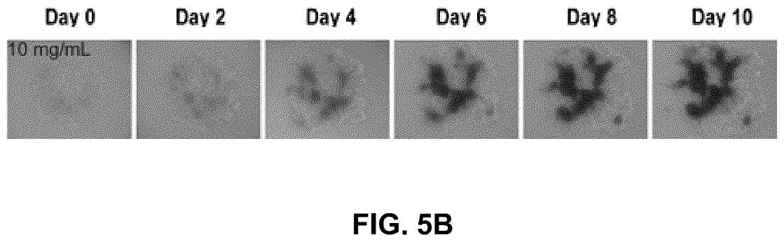

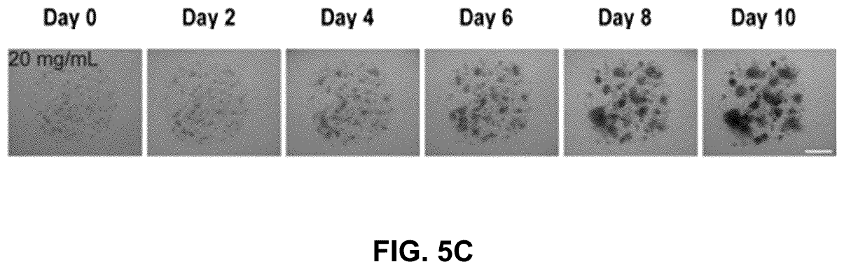

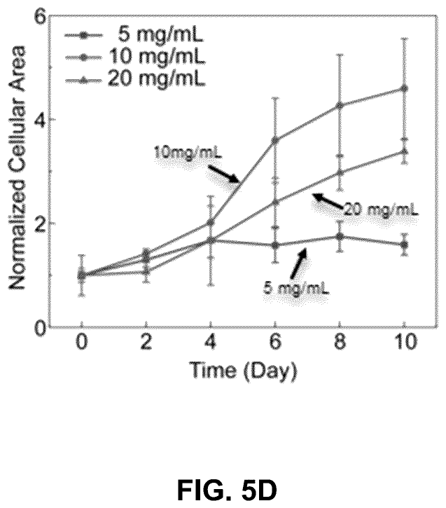

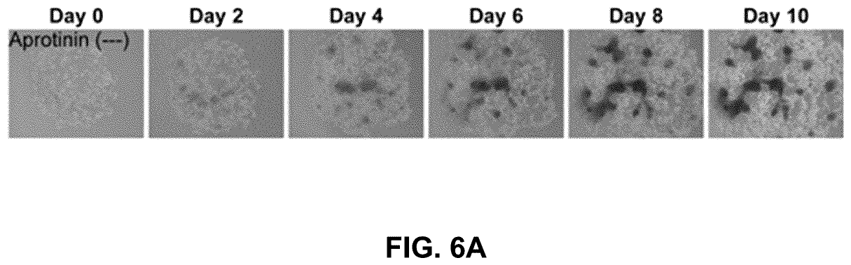

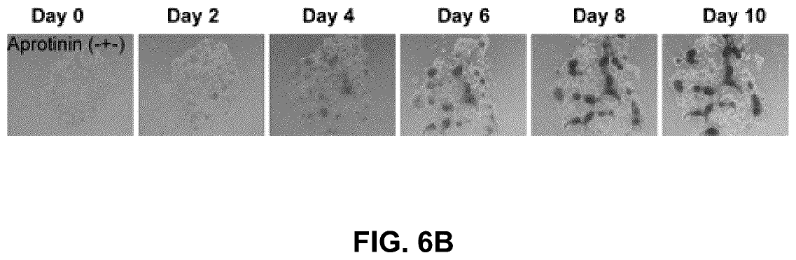

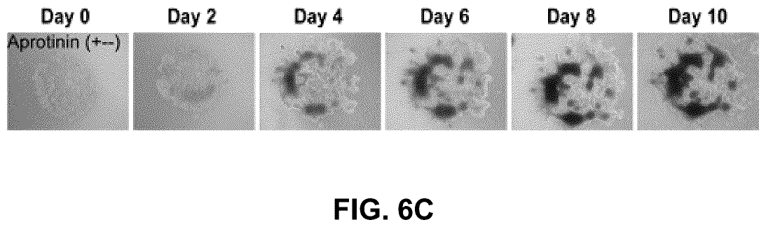

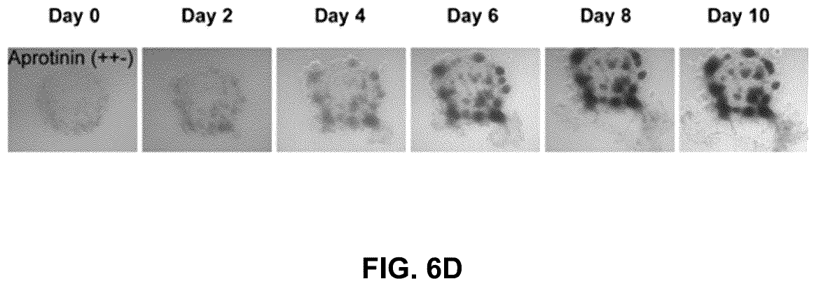

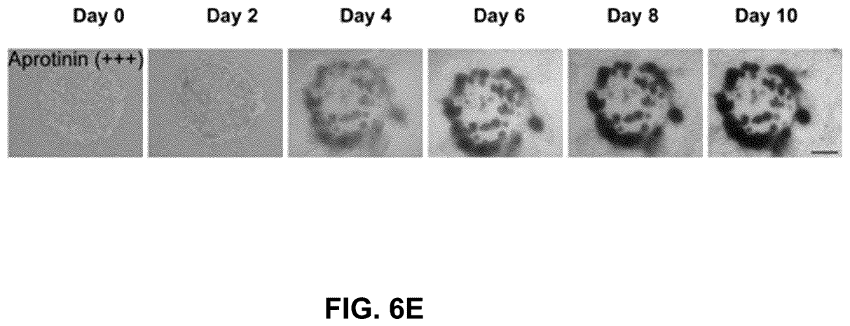

Cellular activities were monitored by capturing time-lapse fluorescence images with confocal microscopy. FIGS. 1A and 1B show the distribution of HaCaT cells over an 8-day period after cells adapted to the 3D hydrogel matrix. From the comparison of cell distribution at each imaging day, two main features of cellular activities were observed, namely, spatial expansion and increased cell population. Both cell migration (FIG. 1C) and proliferation (FIG. 1D) were quantified by analyzing fluorescence intensity of GFP-E-cad expressed by keratinocytes, which provided experimental guidance for the design of multilayered epidermal models in the following steps. As shown in FIGS. 1B and 1C, cells migrated through the surrounding 3D matrix and out from the primary seeding droplet. The leading cells could travel ˜1.4 mm away from the boundary of droplets on day 8. In the meantime, summed GFP-E-cad intensity exhibited greater than threefold enhancement (FIG. 1D), indicating significant cell population increase. The gel formula was optimized to maintain the 3D environment over the test window and to facilitate the activities of encapsulated cells (FIGS. 5A-5D). In addition, aprotinin, an antifibrinolytic protein, was added into the fibrin matrix to tune its degradation rate, promoting a collective migration of keratinocytes in the surrounding fibrin matrix (FIGS. 6A-6E).

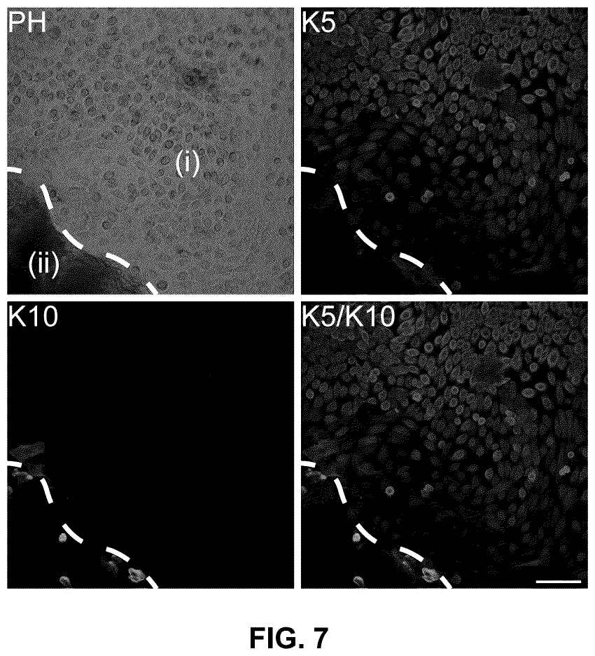

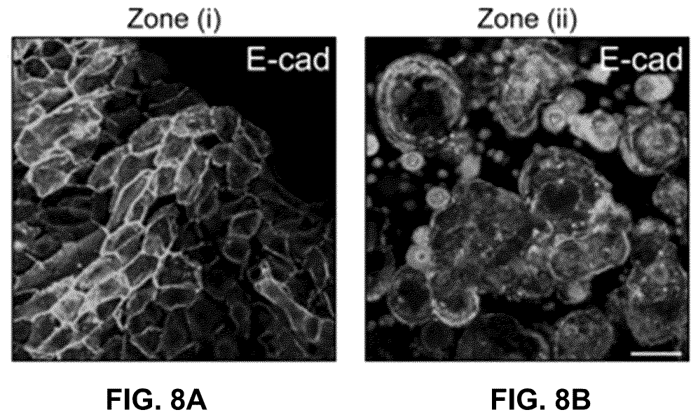

Keratinocyte differentiation is an important process that drives epidermal stratification and maturation. To monitor cellular differentiation, the 3D cultured keratinocytes were characterized by immunostaining of important differentiation markers. The panoramic fluorescence images in FIG. 1E show a representative 3D-cultured sample after staining. The E-cad expression was observed. Cell distribution was observed via conventional DAPI nucleus staining. The signals are generated from the immunostaining of intermediate filament proteins, keratin 5 (K5) and keratin 10 (K10). K5 is abundant in basal keratinocytes. Once a basal keratinocyte differentiates and migrates towards the suprabasal layer, K10s are expressed, which help form a new biomechanical environment. These two keratins can be used to characterize different stages of keratinocyte differentiation. Interestingly, a heterogeneous distribution of K5 and K10 was found over the entire 3D-cultured sample. As illustrated in FIG. 1E, two Zones were observed with distinctive features. As highlighted in FIG. 1F and FIG. 7, zone (i) was formed by the collective migration of keratinocytes that originated from the deposited fibrin droplets. Cells in zone (i) were continuously packed in a monolayer showing a typical epithelial pattern, as observed in the zoom-in view of the GFP-E-cad channel (FIG. 8A), and dominantly expressed K5, indicating their basal status. These collectively migrated cells could serve as the base for the ensuing epidermal regeneration. Compared with zone (i), keratinocytes spontaneously aggregated to numerous cell clusters (FIG. 8B) within the initial droplet (zone (ii)), and meanwhile a higher density of K10 expression (Panel (ii) in FIG. 1F) was observed, suggesting the occurrence of keratinocyte differentiation in some cell clusters that were embedded in the 3D matrix. Although differentiated cells are randomly distributed in the droplets, which could not be used to directly mimic the highly hierarchical structure of the epidermis, the cell-laden droplet did provide sufficient active basal keratinocytes in the 3D microenvironment for the regeneration of a basal layer through collective cell migration. This regional difference was further confirmed by comparing K5- and K10-stained areas in the two zones (FIG. 1G).

### Dynamic Regeneration of Multilayered Epidermal Models

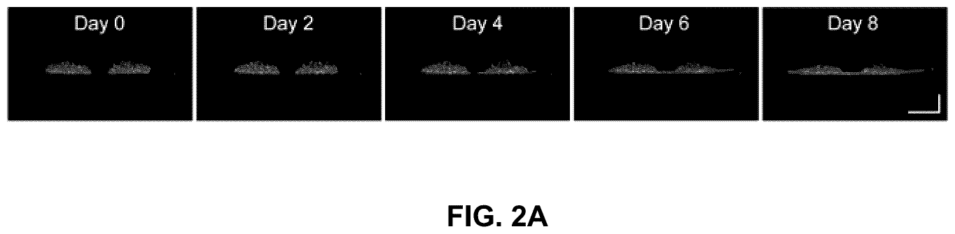

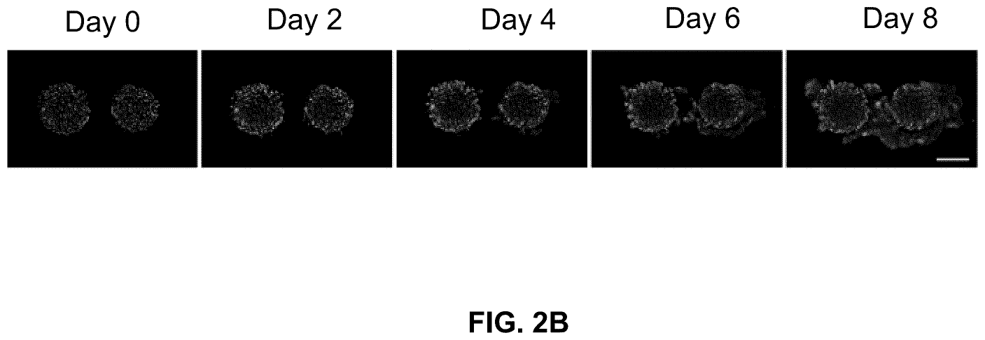

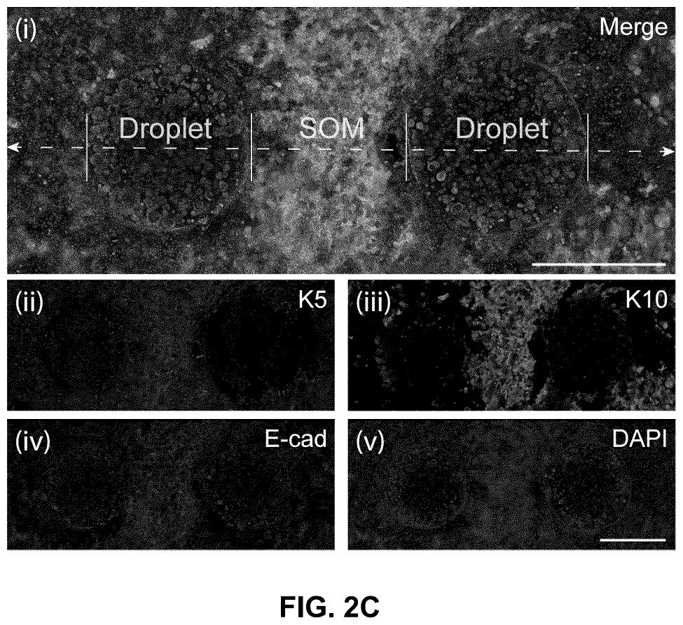

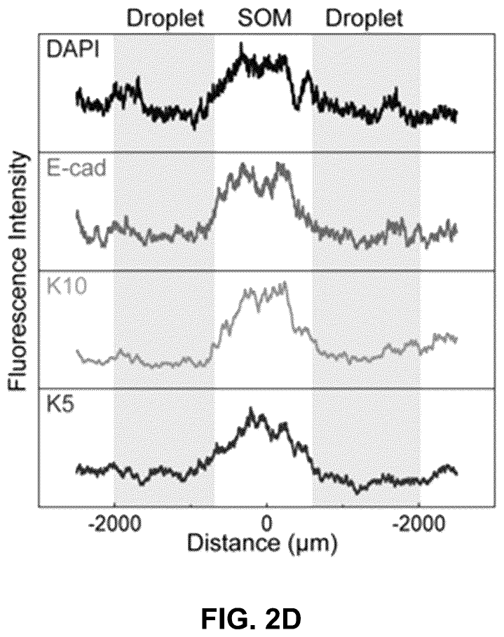

A biofabrication strategy was developed by combining 3D bioprinting of cell-laden fibrin droplets as initial cell sources and postprint cell self-organization to construct multilayered epidermal tissue, as depicted in FIG. 1H. Based on the results discussed in the previous section, two droplets (1.5 μL) of HaCaT cell-seeded fibrin hydrogel were precisely placed onto a bulk 3D fibrin matrix using a custom-built 3D bioprinter [Refs. 34-36]. It has been established that collective cell activities are influenced by the positioning of cell aggregates. Therefore, the inter-droplet distance (d) was selected by taking account of both temporal and spatial efficiency of model construction. As shown in FIGS. 2A and 2B, the edge-to-edge distance was set between 800-1000 μm, which was determined by the observed cell migration rate through the fibrin matrix to allow a 2-week test window and a roughly 1 mm² area for observation of cell behaviors in response to the subsequent treatments. Extremely low numbers of cells (˜3×10³) were used for this fabrication approach, which is more favorable for applications in precision medicine due to the limited availability of patient cells. Time-lapse fluorescence images in FIGS. 2A and 2B show the proliferation and collective migration of encapsulated keratinocytes. These two processes are important cell activities for re-epithelization during skin wound healing. With a longer culture time, cells that migrated from the two droplets merged to form a confluent cell monolayer in the defined interspace. Because of the spatial constraint, these tightly packed cells sought to expand vertically, inducing the SOM, similar to the keratinocyte transformation observed during in vivo epidermal stratification.

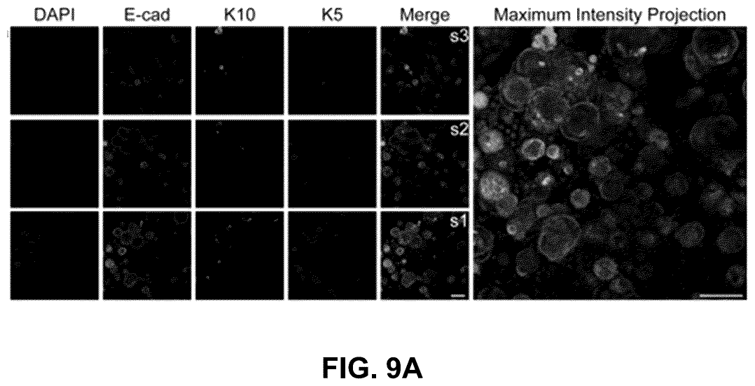

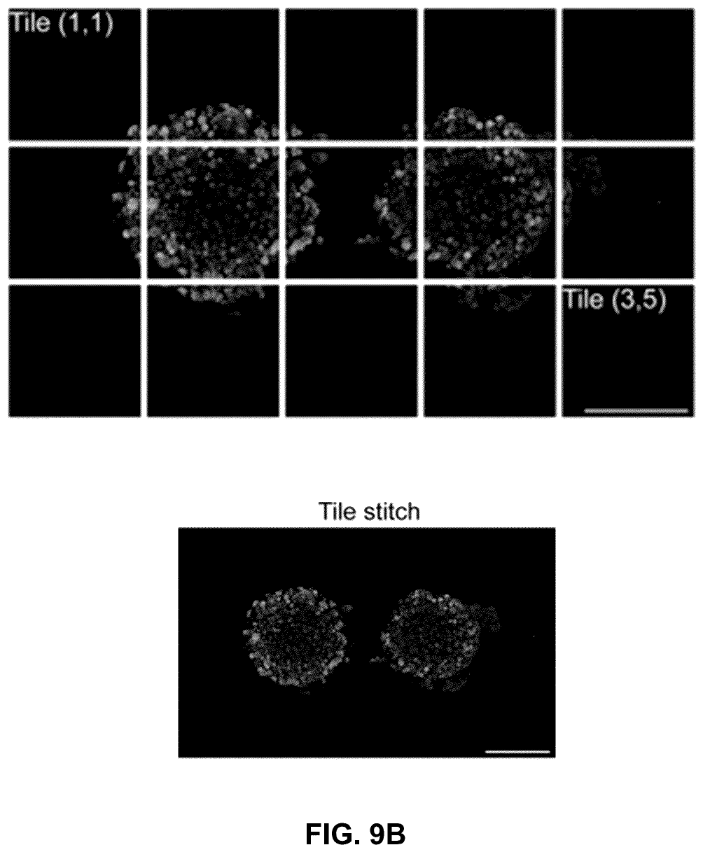

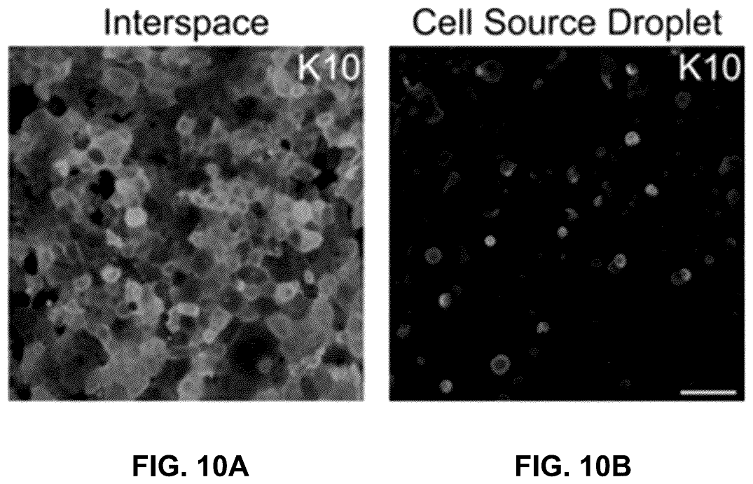

Samples were then fixed and stained to evaluate cell differentiation status, as shown in FIG. 2C and FIGS. 9A and 9B. Moreover, the spatial distribution of each imaged biomarker was mapped along the central axis of the two keratinocyte-seeded droplets, as illustrated in FIG. 2C, by quantifying fluorescence intensity. Both panoramic microscope images (FIG. 2C) and fluorescence intensity plots of four channels (FIG. 2D) show the spatial organization of the 3D tissue constructs. The interspace between the two printed droplets exhibited the highest cell density, as multilayered cell architecture was generated via collective migration of encapsulated HaCaT cells. The strong expression of keratins implied the active re-epithelization through keratinocytes proliferation (K5) and differentiation (K10, FIGS. 10A and 10B) in this region. It also hinted that the tightly packed basal keratinocytes self-initiated differentiation and drove the formation of epidermal SOMs without creating air-liquid-interface. or introducing external Ca²⁺ stimulation. The observed crowding-triggered keratinocyte differentiation was consistent with previous reports. Therefore, this dynamic self-organization of keratinocytes that was guided by the constructed geographical environment could be employed to simulate the formation of skin barriers in vitro.

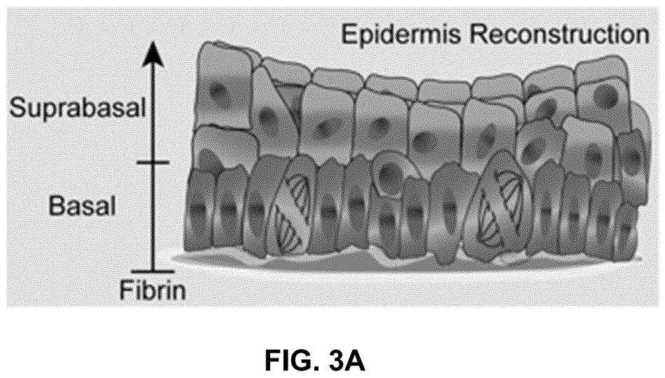

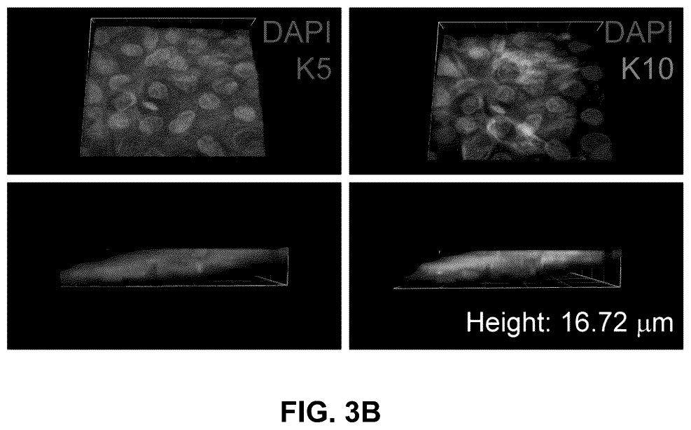

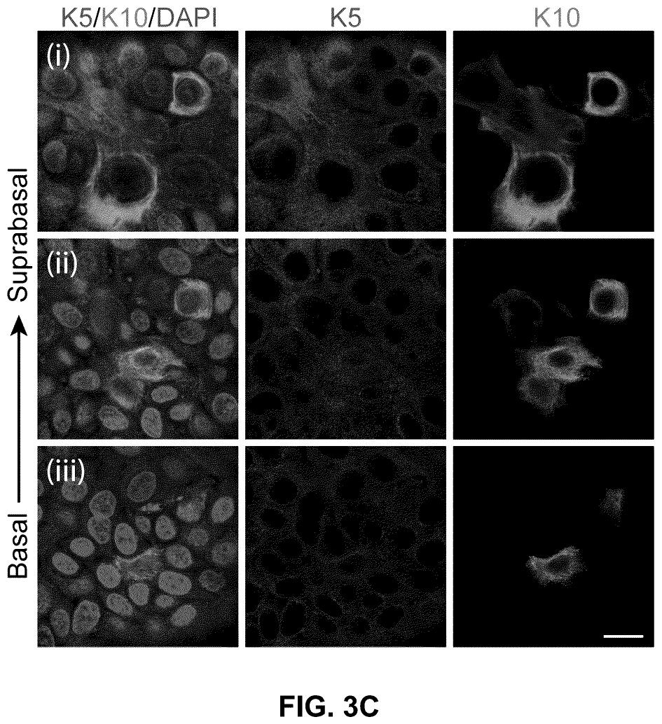

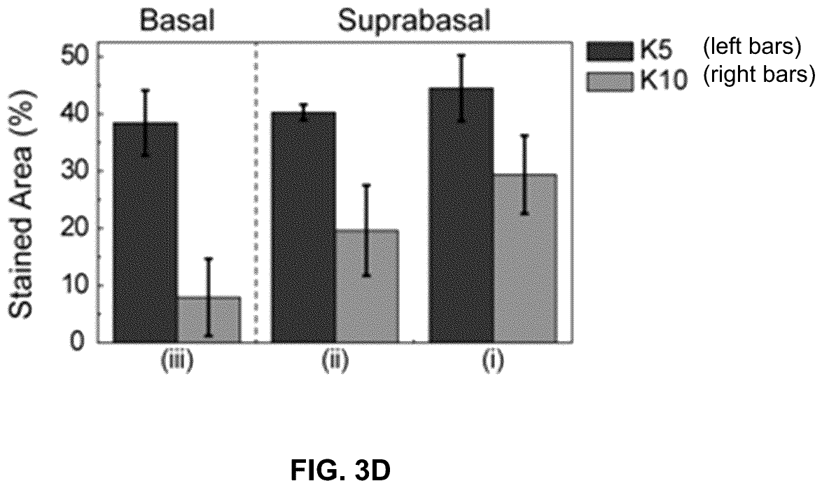

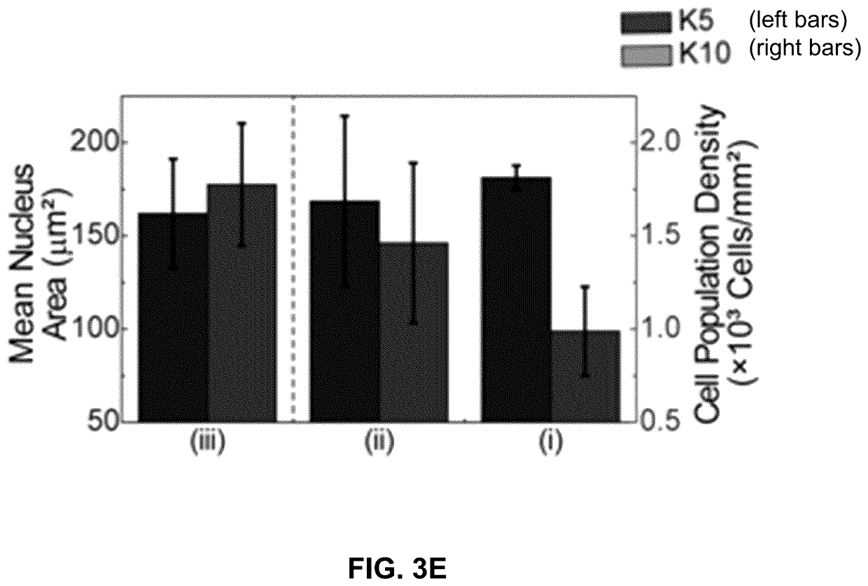

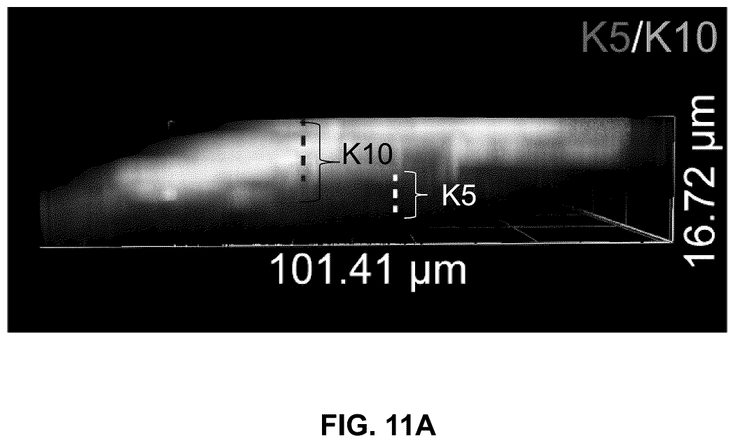

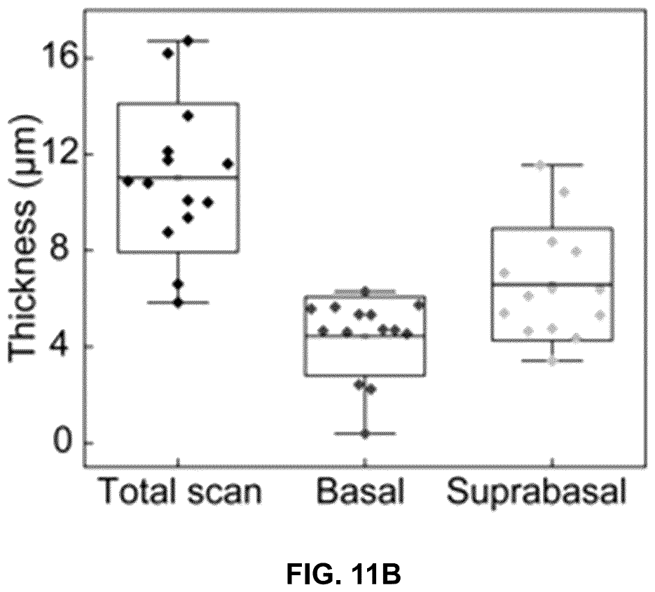

A unique characteristic of the epidermis is its highly hierarchical layer-by-layer cell structure. This structural hierarchy is formed by vertical expansion of keratinocytes initiated at the basal layer. Through asymmetric mitosis, daughter cells of highly proliferative basal keratinocytes migrate toward the suprabasal layers, differentiate to more rigid cells for the protection function of skin, and simultaneously lose their proliferative capability (illustrated in FIG. 3A). To highlight the 3D arrangement of keratinocytes and structural hierarchy in these SOMs, representative regions were scanned with a confocal microscope (FIG. 3B). From DAPI signals, it was observed that cells widely distributed in multiple vertical layers. Similar to previous experiments, intermediate filament proteins, K5 and K10, were stained. As shown in the lateral views of the bottom-up scan (FIG. 3B, bottom row and FIG. 11A), K10 expression that indicates suprabasal status was mainly detected in top layers with a thickness of 6.58±2.32 μm (FIG. 11B) while cells that only expressed K5 were located in the bottom sections (4.44±1.64 μm, FIG. 11B), corresponding to basal layers. The total thickness of the SOMs was measured in the range of 5.84-16.72 μm from 14 scanned regions of three independent samples. The construction of such fine stratified epidermal structures is beyond the capability of conventional 3D bioprinting technologies.

To further investigate differentiation stages of keratinocytes in each layer, fluorescence images were selectively captured of three representative horizontal cross sections following the basal-to-suprabasal direction (FIG. 3C). In contrast to the random spatial distribution of cell differentiation shown in fibrin droplets, K10 expression was steadily enhanced from the bottom basal layer to the upper suprabasal layers (FIG. 3B bottom left panel and FIG. 3D). This gradual vertical alteration of differentiation status in SOMs recaptured the process of bottom-up expansion of basal keratinocytes in vivo. Notably, high K5 expression was observed in all keratinocyte layers in the model (FIG. 3B bottom right panel and FIG. 3D), which is associated with the proliferative capability of epidermal cells. The co-existence of K5 and K10, especially at upper suprabasal layers, could be due to the immortal nature of the HaCaT cell line that was selected in this study. It is also possible that some differentiated keratinocytes have not completed the epidermal morphogenesis.

In addition to keratin expression, cell morphology also exhibited a basal-to-suprabasal transition within the SOMs of fabricated epidermal models, as shown in FIG. 3C. Nucleus size and cell density were quantified by calculating the DAPI-stained area and counting population in each keratinocyte layer, to show this bottom-to-top transition of cellular patterns (FIG. 3E). The three representative layers in SOMs were examined. With the scan moving upward, the mean nucleus area increased by ˜20 μm² from the basal to upper suprabasal layer whereas the population density declined over 45%. Both results could correspond to enlarged cell areas at a horizontal cross section of 3D tissue constructs. These variations in cell morphology match physiological transitions in layers of the epidermis. It has been well established that keratinocytes undergo programmed structural changes during terminal differentiation, and when keratinocytes move up, the upregulated expression of K10 strengthens the cytoskeleton, resulting in cell flattening. Collectively, these results provide solid evidence that SOMs based on 3D cultured keratinocytes could closely mimic the basal-to-suprabasal transition of the epidermis at both phenotypic and genotypic levels.

### Biomimicking the Pathological Microenvironment of PV

Next introduced were cell-cell junction disruptions, and the 3D fabricated epidermal tissues were used as a skin disease model. Specifically, to reconstruct the pathological microenvironment of PV in vitro, anti-Dsg3 antibody (AK23) was added to dissociate the cell-cell junctions by targeting desmosomes in the basal and suprabasal layers. It has been well established that this antibody treatment can induce PV phenotype in both 2D monolayer cultured keratinocytes and animal models. Compared to these previously reported models, the 3D epidermal architectures were designed to provide a deeper insight regarding spatial arrangements and keratinocyte differentiation status.

As shown in FIG. 4A, Dsg3, the main target of AK23, was labeled by a fluorescent dye to visualize desmosomal junctions between keratinocytes in the layered epidermis model. In the untreated samples (FIG. 4A left column), sharp and clear boundaries were observed, indicating the generation of abundant ordered desmosomes. In contrast, wider and incompact junctions were observed in the AK23-treated samples, indicating desmosome disassembly (FIG. 4A right column). To better analyze desmosome disassembly, cell-cell adhesions were quantitatively characterized by the distribution of Dsg3 fluorescence intensity across randomly sampled junctions, as indicated by white lines in FIG. 4A. As shown in FIGS. 4B and 4C, the single-peaked curves of untreated control samples corresponded to intact cell-cell junctions with Dsg3 that tightly concentrated at the boundary of two adjacent cells while a series of discrete peaks (curves) that represent a scattered distribution of Dsg3 were observed at both basal and suprabasal layers after AK23 treatment, which confirmed the dissociation of these desmosomal cadherins. The result of AK23-induced desmosome disassembly is consistent with previous studies on either 2D keratinocyte monolayers or patient-derived histological samples. Intriguingly, two distinct alterations were found of cell-cell junctions at the two epidermal cell layers. In a representative AK23-treated sample, the main Dsg3 peak split into two peaks with a peak-to-peak distance of around 3 μm in the basal layer, whereas a more discrete distribution was observed with a distance range of ≈10 μm at the disrupted junction site in the suprabasal layer. Accompanying the wider separation, Dsg3 intensity in the suprabasal layer decreased between each peak at the dissociated junction, suggesting a more thorough dissociation and potentially a complete loss of cohesion between keratinocytes in comparison to the basal layer.

To reinforce the diverse pathological responses of keratinocytes at basal and suprabasal layers, also compared were their FWHM (FIGS. 12A and 12B), which represents the mean width of cell-cell junctions. Both layers showed increased junction width after AK23 treatment as compared with controls (FIG. 4D) In addition, a greater degree of variation in junction width was detected in the suprabasal layer than the basal layer, indicating a more aggressive process of junction disruption from AK23 antibody treatment. This difference in junction dissociation caused by physiological locations was further confirmed by the significant increase of overall Dsg3 intensity from basal to suprabasal layers (FIG. 4E). This could be attributed to the higher permeability of the looser suprabasal layer, making it more favorable to the immunostaining process. The epidermal models may serve as an in vitro tool to investigate cell behaviors within hierarchical structures of multilayered keratinocytes during the pathogenesis of PV or other skin diseases based on disorders of cell junctions.

### Example 2

### 3D Biofabrication of Comprehensive Skin Models Containing Both Epidermis and Dermis

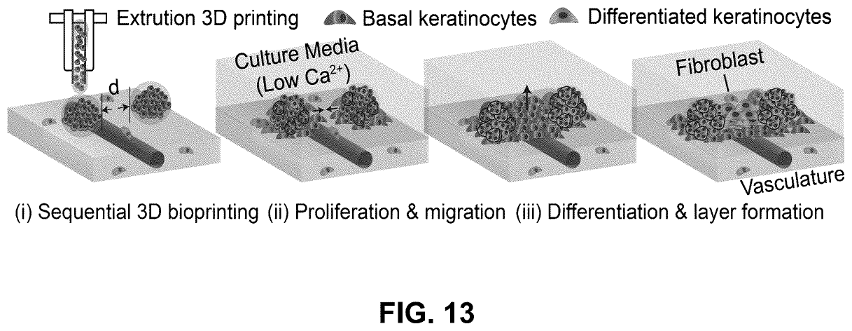

To better mimic the multilayered structure of native skin, keratinocyte-laden fibrin droplets are deposited onto a human dermal fibroblast (HDF)-laden, vascularized fibrin hydrogel. The addition of HDFs and microvascular channels functionalizes the fibrin hydrogel substrate as a dermal layer. The embedded fibroblasts remodel the fibrin hydrogel, significantly increasing the complexity of the dermal ECM in the skin models. The endothelium-lined microchannel acts as a perfusable vascular conduit, facilitating nutrient and gas exchange within the biofabricated tissue structures and mimicking in vivo molecular delivery to investigate therapeutic agent and antibody molecule transport. See FIG. 13.

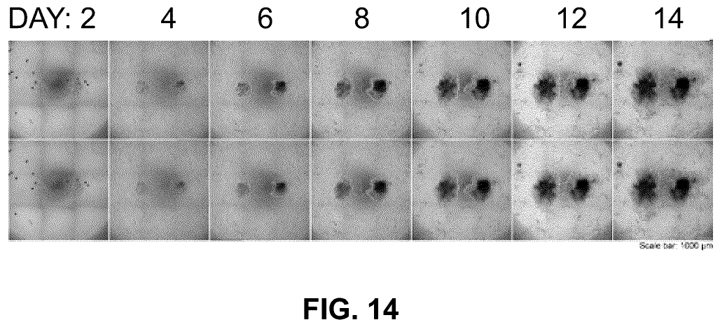

The 3D co-culture of keratinocytes and fibroblasts was conducted to evaluate whether the introduction of fibroblasts would negatively affect the epithelization process. From time-lapse brightfield images, the addition of HDFs in the underlying dermal layer did not affect the proliferation or collective migration of keratinocyte cells on the fibrin substrate. Meanwhile, the fibrin hydrogel supported the proliferation of encapsulated fibroblasts. See FIG. 14.

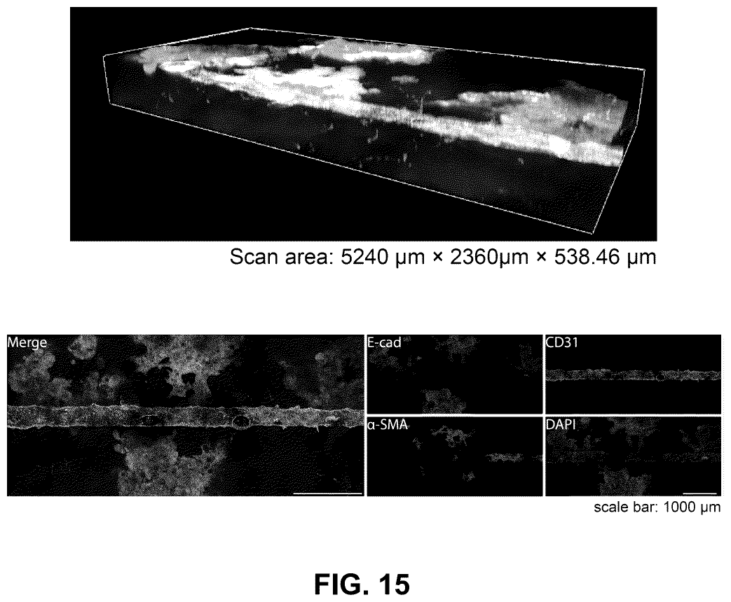

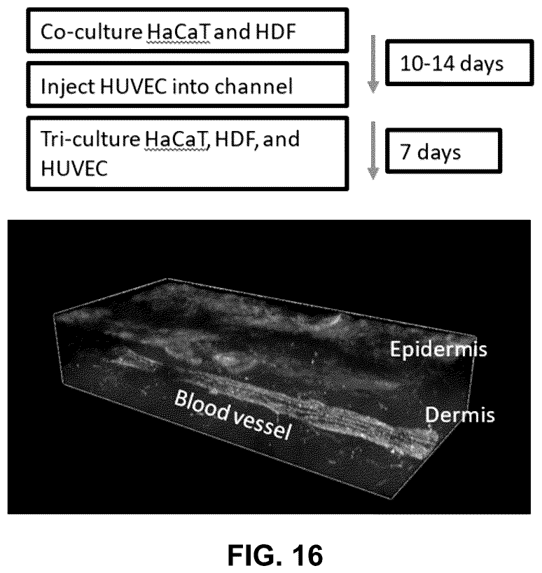

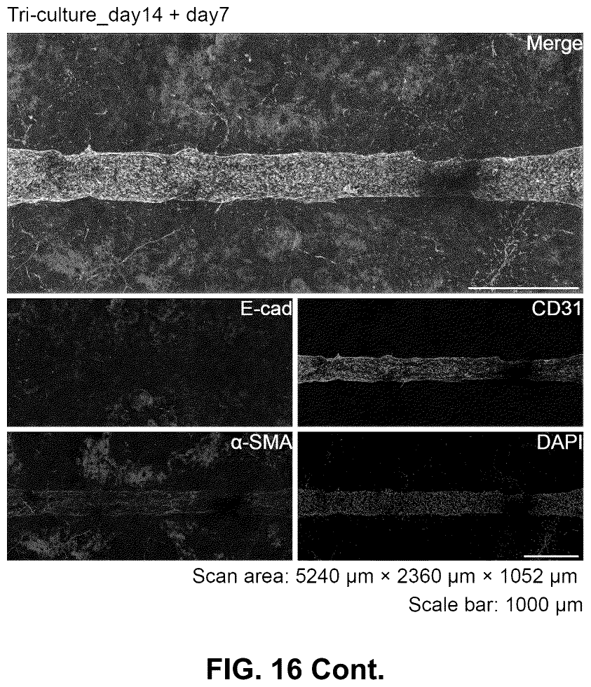

GFP-E-cad-HaCaT cells, as used in Example 1, were utilized for epidermis reconstruction, while primary HDFs and Human umbilical endothelial cells (HUVECs) were integrated into the dermal layer based on their physiological locations, as illustrated in FIG. 13. In this example, four keratinocyte-source droplets were printed to define the interspace for the formation of stratified dermal layers. Cellular activities were dynamically monitored by confocal microscopy after the three cell types were precisely positioned within the 3D tissue architecture. Four biomarkers were used to characterize their spatial locations: 1) E-cad (green-fluorescent E-cadherin), tagged to keratinocytes; 2) α-SMA (a-smooth muscle actin), an activated fibroblast marker; 3) CD31 (platelet endothelial cell adhesion molecule 1), a vascular marker; and 4) DAPI, for overall cell distribution. The intermediate and mature stages of the vascularized skin models were revealed through 3D-reconstructed confocal images and panoramic Z-stacked fluorescence images, as shown in FIGS. 15 and 16, respectively.

As shown in FIGS. 15 and 16, all three cell types were located in positions consistent with native skin structure: fluorescent E-cad, tagged to keratinocytes, was mainly observed on top of the 3D matrix; CD31 was concentrated along the HUVEC-lined microchannel; and α-SMA, scattered throughout the matrix, was associated with embedded fibroblasts. At the intermediate stage, keratinocytes collectively migrated from the droplets and partially occupied the interspace (FIG. 15). Interestingly, α-SMA expression was also detected in the keratinocytes within the interspace but not in those remaining in the source droplets. This may be linked to the high mobility of these migrating keratinocytes, as α-SMA is typically expressed by normal epithelial cells undergoing epithelial-mesenchymal transition during wound healing. At the mature stage, confluent keratinocyte layers fully covered the interspace between the droplets (FIG. 16). α-SMA expression increased significantly, corresponding to further proliferation and activation of the HDFs. Notably, compared to the intermediate stage, more a-SMA signals were detected surrounding the microvascular channel, indicating strong endothelium-stromal interactions in the dermal layer.

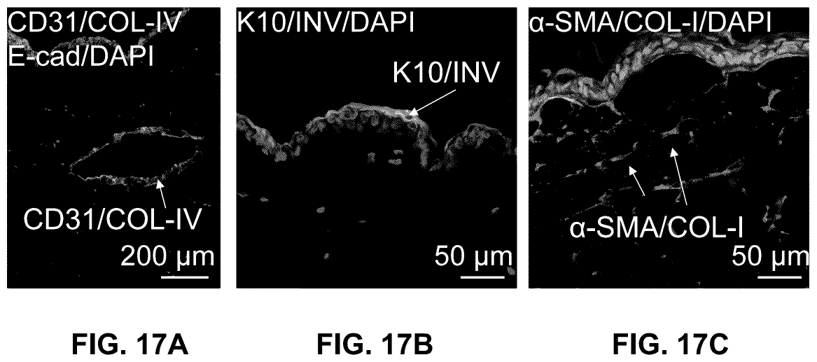

The biofabricated skin tissue models were microsectioned for immunohistochemistry staining to more thoroughly demonstrate their hierarchical epidermal structures and complex vascularized dermal microenvironments. The lumen of the HUVEC-lined microvascular channel within the dermal layer (FIG. 17A) is highlighted by the staining of the vascular marker CD31. Collagen type IV (COL-IV), a component of the basement membrane, was also observed to co-localize with CD31. The co-localization of CD31 and COL-IV suggests the deposition of the basement membrane, promoting endothelial adhesion and the formation of the endothelium within the microchannel to regulate molecular permeation. The tissue sections further exhibit the structural hierarchy of the multilayered epidermis. In addition to the basal-to-suprabasal transition, revealed by K5/K10 expression in Example 1, the formation of late spinous and granular suprabasal layers was characterized by staining another differentiation marker, involucrin (INV), as shown in FIG. 17B. The observation of multiple differentiation states suggests that the biofabricated 3D skin models faithfully recapture the multilayered architecture of native epidermis. Additionally, the dermal matrix was examined by staining for α-SMA and collagen type I (COL-I), the major components of the native dermis (FIG. 17C). While α-SMA expression is associated with HDF distribution and activation, the detection of COL-I confirms fresh ECM protein deposition in the 3D matrices. This fibroblast-induced remodeling significantly enhances the complexity of the dermal ECM, allowing the 3D skin model to more closely recapitulate native skin structure.

### Reestablish Native Routes of Molecular Delivery Using the 3D Biofabricated Skin Model

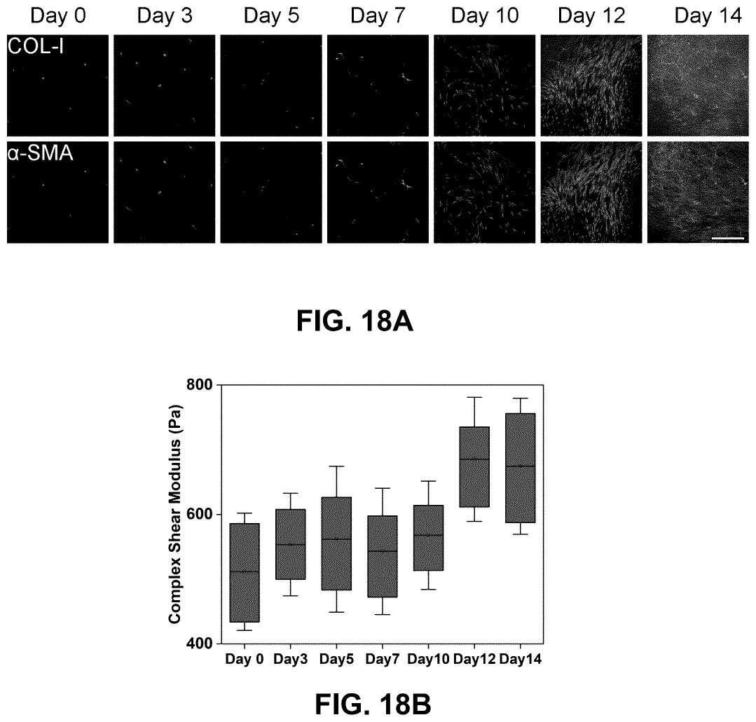

While the distant delivery of functional biomolecules relies on the vascular system, molecular diffusion in local tissue microenvironments is dominated by ECM components and biomechanical properties. Therefore, fibroblast-induced dermal ECM remodeling was further investigated. HDFs embedded within the fibrin matrix were 3D cultured using coculture medium for skin model construction. Over the course of two weeks, the production of COL-I and the expression of α-SMA were characterized in the fibroblast-laden fibrin matrix (FIG. 18A). The upregulated α-SMA expression suggests steady proliferation and activation of HDFs. As a result, collagen fibers were gradually deposited by the activated fibroblasts within the hydrogel matrix. This extensive increase in stromal components intrinsically strengthened the dermal matrix over time, consistent with the results of parallel mechanical testing (FIG. 18B). Nanoindentation measurements showed that the complex shear modulus of the 3D matrices increased by approximately 1.5-fold, quantitatively demonstrating fibroblast-induced dermal ECM remodeling.

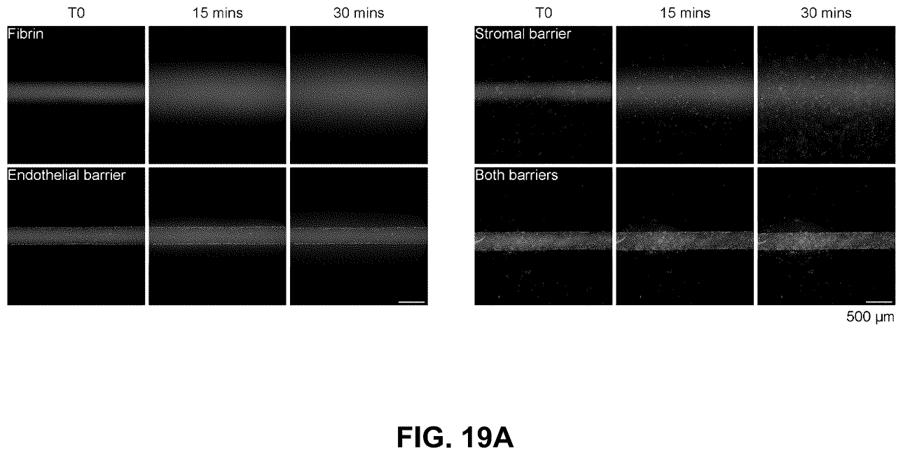

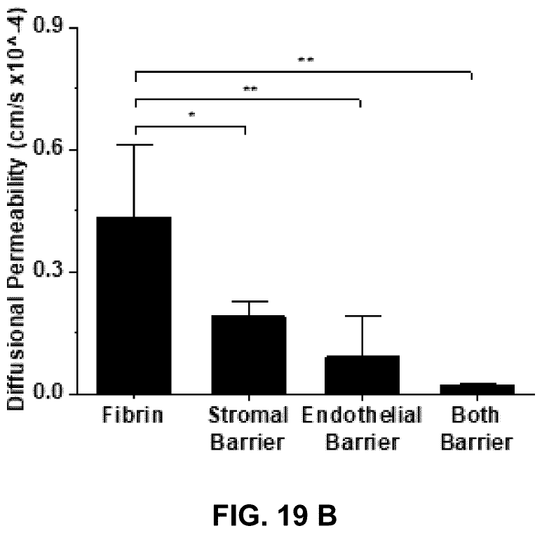

Within these comprehensive skin models, stratified epidermal tissues were supported by a vascularized, fibroblast-laden fibrin hydrogel. Both the vasculature and stromal matrices are important tissue compartments that regulate molecular penetration. To test their tissue barrier function, a fluorescently labeled dextran (150 kDa), matching the molecular weight of Immunoglobulin G (IgG), was dynamically injected into the microchannel. The molecular distribution was monitored by tracking the fluorescent signal. The permeability was quantitatively compared when different tissue barriers were integrated by assessing the dextran fluorescent intensity. Acellular hydrogel matrices and microchannels were employed as controls. Both the resident fibroblast-induced stromal barrier and the endothelial barrier along the microchannel regulated molecular transport. Therefore, the developed 3D skin model can be used to determine the impact of molecular transport routes in studies of autoimmune skin disease pathogenesis and for testing therapeutic agents. See FIG. 19.

### In Vitro Recapitulating PV Pathogenesis

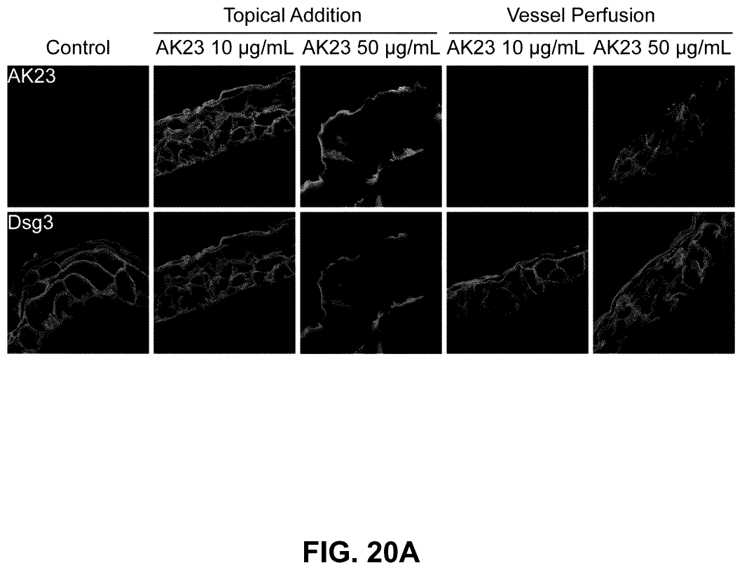

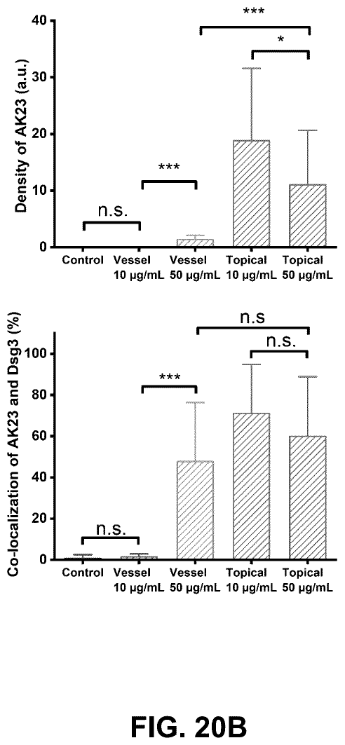

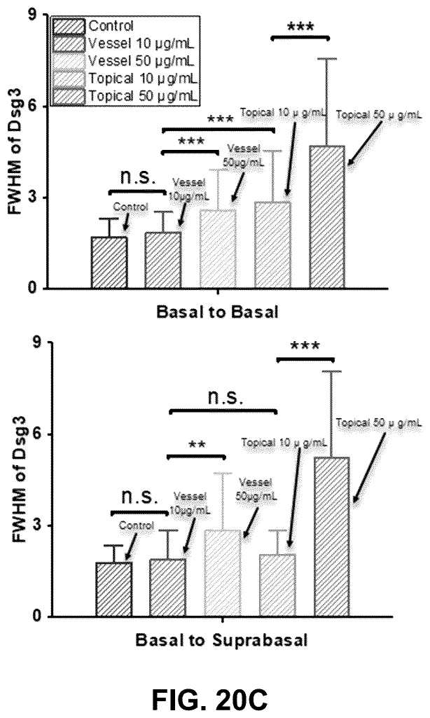

Current PV disease models mainly rely on monolayer-cultured keratinocytes and animal/human skin biopsies, where autoimmune antibodies are directly applied to the epidermis. However, in the human body, these antibodies travel through the bloodstream and then diffuse through the dermal stroma to reach their desmosomal antigens in the stratified epidermis. Unlike conventional skin models, the biofabricated tissue constructs demonstrated here contain an endothelialized microchannel to simulate the in vivo delivery of PV antibodies, and a fibroblast-laden dermal matrix to evaluate their local diffusion. To elucidate the impact of dermal microenvironments on autoimmune antibody penetration in PV pathogenesis, autoimmune antibodies were introduced to the 3D skin model through two treatment routes: 1) topical application, as in conventional PV models, where antibodies are directly applied to the epidermis, and 2) vessel perfusion, where antibodies are injected via the microvascular channel to mimic in vivo molecular delivery. As in Example 1, AK23 was used to dissociate Dsg3 junctions. Two concentrations of AK23 (10 and 50 μg/mL) were applied to the models for 24 hours, while an untreated skin construct was used as a healthy control. See FIG. 20.

To characterize PV pathogenesis, tissue models representing each experimental condition were microsectioned and immunostained (FIG. 20A) to visualize AK23 distribution (top row) and Dsg3 cell-cell junctions (bottom row). To assess the interactions between the autoimmune antibodies and their antigens at cell-cell junctions, the fluorescent intensity and spatial locations of AK23 and Dsg3 were quantified, as shown in FIG. 20B. While AK23 specifically targets Dsg3 junctions, its access to the epidermis was significantly limited by the intrinsic barrier function of the dermal stroma when comparing vessel injection to direct application. Only when a high concentration of AK23 (50 μg/mL) was introduced via the microvascular channel could an efficient concentration of antibodies be detected in the stratified epidermal layers.

Following the four treatment conditions, the skin constructs exhibited distinct disease phenotypes. Using the same quantification method as in Example I, FWHM values were measured to evaluate desmosomal dissociation (FIG. 20C). When the epidermis was directly exposed to AK23, Dsg3 junctions were readily disassembled. In cases where a high concentration of AK23 was applied, total loss of Dsg3 throughout the epidermal layers was observed in some PV models. However, when the AK23 concentration was decreased, Dsg3 junctions between parallel basal cells were primarily affected. A possible explanation for this phenomenon is that the upper stratum granulosum layer (which expresses involucrin, INV) contains lipids that form a waterproof barrier, causing the AK23 solution to primarily reach the basal layer at the bottom of the epidermis first.

When AK23 was delivered via the microvascular channel and traveled through the dermal matrix, a much lower concentration of AK23 reached the epidermis. As a result, Dsg3 dissociation was primarily observed in the basal and adjacent suprabasal layers. Consistent with the AK23 distribution assessment, no junction disassembly was observed in the skin model treated with 10 μg/mL AK23 through the microvascular channel. These results demonstrate the significance of the dermal microenvironment in skin disease modeling and suggest that the 3D skin model presented here will be a promising in vitro tool for understanding skin disease development and for screening/repurposing therapeutic agents.

### REFERENCES

[1] B. Alberts, A. Johnson, J. Lewis, D. Morgan, M. Raff, K. Roberts, P. Walter, Molecular Biology of the Cell, 6th ed., Garland Science, New York, NY, USA 2015.
[2] J. E. Lai-Cheong, K. Arita, J. A. McGrath, J. Invest. Dermatol. 2007, 127, 2713.
[3] M. Hegazy, A. L. Perl, S. A. Svoboda, K. J. Green, Annu. Rev. Pathol.: Mech. Dis. 2022, 17, 47.
[4] M. Kasperkiewicz, C. T. Ellebrecht, H. Takahashi, J. Yamagami, D. Zillikens, A. S. Payne, M. Amagai, Nat. Rev. Dis. Primers 2017, 3, 1.
[5] C. L. Simpson, D. M. Patel, K. J. Green, Nat. Rev. Mol. Cell Biol. 2011, 12, 565.
[6] T. Sajda, A. A. Sinha, Front. Immunol. 2018, 9, 692.
[7] K. Seiffert-Sinha, R. Yang, C. K. Fung, K. W. Lai, K. C. Patterson, A. S. Payne, N. Xi, A. A. Sinha, PLOS One 2014, 9, e106895.
[8] C. K. M. Fung, K. Seiffert-Sinha, K. W. C. Lai, R. Yang, D. Panyard, J. Zhang, N. Xi, A. A. Sinha, Nanomed. Nanotechnol. Biol. Med. 2010, 6, 191.
[9] X. Jin, J. Rosenbohm, E. Kim, A. M. Esfahani, K. Seiffert-Sinha, J. K. Wahl III, J. Y. Lim, A. A. Sinha, R. Yang, Adv. biology 2021, 5, 2000159.
[10] M. J. Randall, A. Jüngel, M. Rimann, K. Wuertz-Kozak, Front. Bioeng. Biotechnol. 2018, 6, 154.
[11] J. Reichelt, Europ. J. Cell Biol. 2007, 86, 807.
[12] L. Koch, A. Deiwick, S. Schlie, S. Michael, M. Gruene, V. Coger, D. Zychlinski, A. Schambach, K. Reimers, P. M. Vogt, B. Chichkov, Biotechnol. Bioeng. 2012, 109, 1855.
[13] Y. Shi, T. L. Xing, H. B. Zhang, R. X. Yin, S. M. Yang, J. Wei, W. J. Zhang, Biomed. Mater. 2018, 13, 035008.
[14] S. Ipponjima, T. Hibi, T. Nemoto, PLOS One 2016, 11, e0163199.
[15] A. K. Miri, I. Mirzaee, S. Hassan, S. Mesbah Oskui, D. Nieto, A. Khademhosseini, Y. S. Zhang, Lab Chip 2019, 19, 2019.
[16] K. J. Green, C. L. Simpson, J. Invest. Dermatol. 2007, 127, 2499.
[17] C. M. Hammers, J. R. Stanley, Annu. Rev. Pathol. 2016, 11, 175.
[18] V. Spindler, J. Waschke, Front. Immunol. 2018, 9, 136.
[19] M. Hospodiuk, M. Dey, D. Sosnoski, I. T. Ozbolat, Biotechnol. Adva. 2017, 35, 217.
[20] X. Wang, S. Wang, B. Guo, Y. Su, Z. Tan, M. Chang, J. Diao, Y. Zhao, Y. Wang, Cell Death & Dis. 2021, 12, 35.
[21] M. Bacakova, J. Musilkova, T. Riedel, D. Stranska, E. Brynda, L. Bacakova, M. Zaloudkova, Int. J. Nanomed. 2016, 11, 771.
[22] M. Robinson, S. Douglas, S. M, Willerth, Sci. Rep. 2017, 7, 6250.
[23] K. Kobayashi, Y. Ichihara, N. Tano, L. Fields, N. Murugesu, T. Ito, C. Ikebe, F. Lewis, K. Yashiro, Y. Shintani, R. Uppal, K. Suzuki, Sci. Rep. 2018, 8,9448.
[24] J. Liu, G. Chen, H. Xu, K. Hu, J. Sun, M. Liu, F. Zhang, N. Gu, NPG Asia Mater. 2018, 10, 827.
[25] T. Hoppenbrouwers, B. Tuk, E. M. Fijneman, M. P. de Maat, J. W. van Neck, Thromb Res. 2017, 151, 36.
[26] M. Persinal-Medina, S. Llames, M. Chacón, N. Vázquez, M. Pevida, I. Alcalde, S. Alonso-Alonso, L. M. Martínez-Lopez, J. Merayo-Lloves, A. Meana, Int. J. Mol. Sci. 2022, 23, 4837.
[27] P. Martin, Science 1997, 276, 75.
[28] R. A. Clark, Ann. N. Y. Acad. Sci. 2001, 936, 355.
[29] K. J. Kearney, R. A. S. Ariens, F. L. Macrae, Semin. Thromb. Hemost. 2022, 48, 174.
[30] K. M. Lorentz, S. Kontos, P. Frey, J. A. Hubbell, Biomaterials 2011, 32, 430.
[31] R. S. Moreci, T. Lechler, Curr. Biol. 2020, 30, R144.
[32] Y. A. Miroshnikova, H. Q. Le, D. Schneider, T. Thalheim, M. Rubsam, N. Bremicker, J. Polleux, N. Kamprad, M. Tarantola, I. Wang, M. Balland, C. M. Niessen, J. Galle, S. A. Wickstrom, Nat. Cell Biol. 2018, 20, 69.
[33] P. Cai, Z. Li, E. S. Keneth, L. Wang, C. Wan, Y. Jiang, B. Hu, Y.-L. Wu, S. Wang, C. T. Lim, E. V. Makeyev, S. Magdassi, X. Chen, Adv. Mater. 2019, 31, 1900514.
[34] F. Meng, C. M. Meyer, D. Joung, D. A. Vallera, M. C. McAlpine, A. Panoskaltsis-Mortari, Adv. Mater. 2019, 31, 1806899.
[35] M. K. Gupta, F. Meng, B. N. Johnson, Y. L. Kong, L. Tian, Y.-W. Yeh, N. Masters, S. Singamaneni, M. C. McAlpine, Nano Lett. 2015, 15, 5321.
[36] D. Joung, V. Truong, C. C. Neitzke, S.-Z. Guo, P. J. Walsh, J. R. Monat, F. Meng, S. H. Park, J. R. Dutton, A. M. Parr, M. C. McAlpine, Adv. Fun. Mater. 2018, 28, 1801850.
[37] B. Ayan, D. N. Heo, Z. Zhang, M Dey, A. Povilianskas, C. Drapaca, I. T. Ozbolat, Sci. Adv. 2020, 6, eaaw5111.
[38] B. S. Kim, W.-W. Cho, G. Gao, M. Ahn, J. Kim, D.-W. Cho, Small Methods 2021, 5, 2100072.
[39] M. Poujade, E. Grasland-Mongrain, A. Hertzog, J. Jouanneau, P. Chavrier, B. Ladoux, A. Buguin, P. Silberzan, Proc. Natl. Acad. Sci. 2007, 104, 15988.
[40] E. Proksch, J. M. Brandner, J.-M. Jensen, Exp. Dermatol. 2008, 17, 1063.
[41] M. Prunieras, M. Régnier, D. Woodley, J Invest Dermatol. 1983, 81, 28s.
[42] D. D. Bikle, Z. Xie, C. L. Tu, Expert Rev. Endocrinol. Metab. 2012, 7, 461.
[43] I. Colombo, E. Sangiovanni, R. Maggio, C. Mattozzi, S. Zava, Y. Corbett, M. Fumagalli, C. Carlino, P. A. Corsetto, D. Scaccabarozzi, S. Calvieri, A. Gismondi, D. Taramelli, M. Dell'Agli, Mediators Inflamm. 2017, 7435621.
[44] C. Blanpain, E. Fuchs, Nat. Rev. Mol. Cell Biol. 2009, 10, 207.
[45] H. H. Bragulla, D. G. Homberger, J. Anatomy 2009, 214, 516.
[46] M. R. Gdula, K. Poterlowicz, A. N. Mardaryev, A. A. Sharov, Y. Peng, M. Y. Fessing, V. A. Botchkarev, J. Invest. Dermatol. 2013, 133, 2191.
[47] K. Tsunoda, T. Ota, M. Saito, T. Hata, A. Shimizu, A. Ishiko, T. Yamada, T. Nakagawa, A. P. Kowalczyk, M. Amagai, Am. J. Pathol. 2011, 179, 795.
[48] E. Walter, F. Vielmuth, L. Rotkopf, M. Sárdy, O. N. Horváth, M. Goebeler, E. Schmidt, R. Eming, M. Hertl, V. Spindler, Sci. Rep. 2017, 7, 1.
[49] D. Egu, A. Sigmund, E. Schmidt, V. Spindler, E. Walter, J. Waschke, Br. J. Dermatol. 2020, 182, 987.
[50] B. J. Roberts, R. A. Svoboda, A. M. Overmiller, J. D. Lewis, A. P. Kowalczyk, M. G. Mahoney, K. R. Johnson, J. K. Wahl, J. Biol. Chem. 2016, 291, 24857.
[51] H. Zhai, X. Jin, G. Minnick, J. Rosenbohm, M. A. H. Hafiz, R. Yang, F. Meng, Small Sci. 2022, 2, 2200051.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of generating a three-dimensional skin tissue construct comprising a basal-to-suprabasal transition, the method comprising:
(a) depositing onto a substrate a droplet comprising one or more undifferentiated keratinocyte cells;
(b) depositing onto the substrate another droplet comprising one or more additional undifferentiated keratinocyte cells; and
(c) maintaining the undifferentiated keratinocyte cells on the substrate in a condition that promotes differentiation of undifferentiated keratinocyte cells;
wherein the droplet of (b) is deposited a predetermined distance away from the droplet of (a);
wherein undifferentiated keratinocyte cells migrate from droplets of (a) and (b), generating a basal cell layer;
wherein proliferation and differentiation of keratinocyte cells of the basal cell layer generate differentiated suprabasal keratinocyte cells and a basal-to-suprabasal transition between at least a subset of adjacent keratinocyte cells of the basal layer and differentiated suprabasal keratinocyte cells; and
wherein the extent of differentiation of keratinocyte cells and the extent of the basal-to suprabasal transition present after (c) varies based on the predetermined distance.

2. The method of claim 1, wherein the predetermined distance is 800 to 1000 micrometers measured edge-to-edge from the droplets of (a) and (b) deposited on the substrate.

3. The method of claim 1, wherein maintaining the undifferentiated keratinocyte cells during (c) comprises maintaining the undifferentiated keratinocyte cells in low-calcium medium.

4. The method of claim 1, wherein the droplets of (a) and (b) each comprise a hydrogel comprising one or more undifferentiated keratinocyte cells.

5. The method of claim 1, wherein the substrate comprises a hydrogel.

6. The method of claim 1, wherein the substrate does not comprise a lattice structure.

7. The method of claim 2, wherein maintaining the undifferentiated keratinocyte cells during (c) comprises maintaining the undifferentiated keratinocyte cells in low-calcium medium;
wherein the droplets of (a) and (b) each comprise a hydrogel comprising the one or more undifferentiated keratinocyte cells;
wherein the substrate comprises a hydrogel; and
wherein the substrate does not comprise a lattice structure.

8. The method of claim 1, wherein the substrate comprises one or more human dermal fibroblast cells.

9. The method of claim 1, wherein the substrate comprises one or more microvascular channels.

10. The method of claim 7, wherein the substrate comprises one or more human dermal fibroblast cells.

11. The method of claim 7, wherein the substrate comprises one or more microvascular channels.

12. The method of claim 10, wherein the substrate comprises one or more microvascular channels.

13. A method of testing an effect of a pharmaceutical on skin tissue, the method comprising:
(1) generating a three-dimensional skin tissue construct comprising a basal-to-suprabasal transition according to the method of claim 1;
(2) contacting the three-dimensional skin tissue construct of (1) with a pharmaceutical; and
(3) observing a change of the three-dimensional skin tissue construct after (2).

14. A method of testing an effect of a pharmaceutical on skin tissue, the method comprising:
(1) generating a three-dimensional skin tissue construct comprising a basal-to-suprabasal transition according to the method of claim 7;
(2) contacting the three-dimensional skin tissue construct of (1) with a pharmaceutical; and
(3) observing a change of the three-dimensional skin tissue construct after (2).

15. A method of testing an effect of a pharmaceutical on skin tissue, the method comprising:
(1) generating a three-dimensional skin tissue construct comprising a basal-to-suprabasal transition according to the method of claim 12;
(2) contacting the three-dimensional skin tissue construct of (1) with a pharmaceutical; and
(3) observing a change of the three-dimensional skin tissue construct after (2).

16. The method of claim 15, wherein the contacting of (2) comprises delivering the pharmaceutical by one or more microvascular channels in the substrate.

17. A method of generating a disease-state model of skin tissue, the method comprising:
(1) generating a three-dimensional skin tissue construct comprising a basal-to-suprabasal transition according to the method of claim 1; and
(2) contacting the three-dimensional skin tissue construct of (1) with an agent to alter the construct to mimic a disease state.

18. The method of claim 17, wherein maintaining the undifferentiated keratinocyte cells during (c) comprises maintaining the undifferentiated keratinocyte cells in low-calcium medium;
wherein the droplets of (a) and (b) each comprise a hydrogel comprising the one or more undifferentiated keratinocyte cells;
wherein the substrate comprises a hydrogel; and
wherein the substrate does not comprise a lattice structure.

19. The method of claim 18, wherein the substrate comprises one or more microvascular channels.

20. The method of claim 19, wherein the contacting of (2) comprises delivering the agent by one or more microvascular channels in the substrate.
